(19) United States
(12) Patent Application Publication
(10) Pub. No.: US 2021/0038204 A1
(43) Pub. Date: Feb. 11, 2021

(54) PULMONARY BIOPSY DEVICES

(71) Applicant: Boston Scientific Scimed, Inc., Maple Grove, MN (US)

(72) Inventors: John A. HINGSTON, FRAMINGHAM, MA (US); Daniel R. QUINN, LITTLETON, MA (US); Steven E. WALAK, NATICK, MA (US); Jason MORRELL, HOLLISTON, MA (US); Michelle Pham TRAN, WILIMINGTON, MA (US); Pat S. PHONGSAVANH, BLACKSTONE, MA (US); Mahfuza AHMED, BROOKLINE, MA (US); Paul SMITH, SMITHFIELD, RI (US); Michael M. BOREK, HOLDEN, MA (US); Gerald FREDRICKSON, WESTFORD, MA (US); Michael E. ZUPKOFSKA, ROCKLAND, MA (US); Douglas C. SHEPARD, MANSFIELD, MA (US); Gene T. STORBECK, MILLIS, MA (US); Michael D. SINISI, BROOKLINE, MA (US)

(73) Assignee: Boston Scientific Scimed, Inc.

(21) Appl. No.: 17/072,257

(22) Filed: Oct. 16, 2020

Related U.S. Application Data
(63) Continuation of application No. 15/082,834, filed on Mar. 28, 2016.
(60) Provisional application No. 62/141,610, filed on Apr. 1, 2015.

(51) Int. Cl. A61B 10/04; A61B 10/02; A61B 1/015; A61M 25/10; A61B 8/12; A61B 8/00

(52) U.S. Cl. CPC A61B 10/04; A61B 10/0233; A61B 1/015; A61B 10/0283; A61M 25/10; A61B 1/00082; A61B 8/4254; A61B 8/445; A61B 8/4494; A61M 2210/1039; A61B 8/12

(57) ABSTRACT

Methods, devices, and systems for obtaining a navigating to a targeted biopsy region are disclosed. An example device for navigating to a targeted biopsy region may include an elongate shaft having a proximal end, a distal end, an inflation lumen, and a vacuum lumen. An inflatable balloon may be positioned proximal to the distal end of the elongate shaft. The device may further include an ultrasound transducer positioned adjacent to the distal end of the elongate shaft and a power and control unit in electrical communication with the ultrasound transducer. A vacuum source may be in fluid communication with the vacuum lumen.

### PRIORITY CLAIM

This application is a continuation of 15/082,834, filed Mar. 28, 2016, which claims priority to U.S. Provisional Patent Application Ser. No. 62/141,610 filed Apr. 1, 2015; the disclosure of which is incorporated herewith by reference

### TECHNICAL FIELD

The present disclosure pertains to medical devices, and methods for manufacturing and/or using medical devices. More particularly, the present disclosure pertains to obtaining a biopsy sample from the body.

### BACKGROUND

A wide variety of medical devices have been developed for medical use, for example, pulmonary use. Some of these devices include catheters, stents, diagnostic tools, and the like, and delivery devices and/or systems used for delivering such devices. These devices are manufactured by any one of a variety of different manufacturing methods and may be used according to any one of a variety of methods. Of the known medical devices, delivery system, and methods, each has certain advantages and disadvantages. There is an ongoing need to provide alternative medical devices and delivery devices as well as alternative methods for manufacturing and using medical devices and delivery devices.

### SUMMARY

This disclosure provides design, material, manufacturing method, and use alternatives for medical devices, including biopsy devices and methods. Example ultrasound biopsy devices and methods are disclosed.

An example forward viewing ultrasound device may comprise:

an elongate shaft having a proximal end, a distal end, an inflation lumen, and a vacuum lumen;

an inflatable balloon positioned proximal to the distal end of the elongate shaft;

an ultrasound transducer positioned adjacent to the distal end of the elongate shaft;

a power and control unit in electrical communication with the ultrasound transducer; and

a vacuum source in fluid communication with the vacuum lumen.

Alternatively or additionally to any of the embodiments above, wherein the ultrasound transducer is positioned to direct acoustic energy distal of the distal end of the elongate shaft.

Alternatively or additionally to any of the embodiments above, wherein when in an inflated state, the inflatable balloon is configured to fill a gap between an outer surface of the balloon and an inner surface a body lumen.

Alternatively or additionally to any of the embodiments above, wherein when the balloon is in an inflated state, the vacuum source is activated to pull a vacuum on the body lumen.

Alternatively or additionally to any of the embodiments above, wherein when the vacuum is present in the body lumen, the power and control unit is activated to supply electrical energy to the ultrasound transducer to generate and direct acoustic energy distal of the distal end of the elongate shaft.

Alternatively or additionally to any of the embodiments above, wherein when the vacuum is present in the body lumen, an infusion fluid is pumped into the body lumen.

Alternatively or additionally to any of the embodiments above, further comprising one or more steering wires disposed within the elongate shaft.

Alternatively or additionally to any of the embodiments above, further comprising an infusion lumen.

Alternatively or additionally to any of the embodiments above, further comprising at least one radiopaque marker.

Alternatively or additionally to any of the embodiments above, wherein the steering wire comprises a strand of polymer fiber twisted with a conductor into a coiled structure.

Alternatively or additionally to any of the embodiments above, wherein the steering wire comprises a plurality of strands of polymer fibers twisted with a conductor into a coiled structure.

Alternatively or additionally to any of the embodiments above, wherein applying heat to the electrical conductor causes a length of the coiled structure to contract.

Alternatively or additionally to any of the embodiments above, wherein applying electricity to the electrical conductor causes a length of the coiled structure to contract.

Alternatively or additionally to any of the embodiments above, wherein the coiled structure comprises a tightly wound coil.

Alternatively or additionally to any of the embodiments above, wherein the coiled structure comprises a loosely wound coil.

Alternatively or additionally to any of the embodiments above, wherein the steering wire comprises a bimetallic strip.

An example ultrasound device may comprise:

an elongate shaft having a proximal end, a distal end, and a lumen extending therethrough;

a penetrating element positioned adjacent to the distal end of the elongate shaft, the penetrating element configured to penetrate tissue;

an ultrasound transducer positioned proximal to the distal tip and adjacent to the distal end of the elongate shaft; and

a power and control unit in electrical communication with the ultrasound transducer.

Alternatively or additionally to any of the embodiments above, wherein the penetrating element has a conical shape.

Alternatively or additionally to any of the embodiments above, wherein the penetrating element has a first sloped side and a second side extending generally parallel to a longitudinal axis of the elongate shaft.

Alternatively or additionally to any of the embodiments above, wherein the penetrating element is slidably disposed within the lumen of the elongate shaft.

Alternatively or additionally to any of the embodiments above, wherein the penetrating element comprises a biopsy needle.

Alternatively or additionally to any of the embodiments above, wherein the ultrasound transducer is embedded in a wall of the elongate shaft.

Alternatively or additionally to any of the embodiments above, wherein the penetrating element has a lumen extending therethrough.

Alternatively or additionally to any of the embodiments above, wherein the penetrating element has a generally flat shape.

Alternatively or additionally to any of the embodiments above, wherein the penetrating element has a first cross sectional size and shape at a first point and tapers distally to a second smaller cross-sectional size.

Alternatively or additionally to any of the embodiments above, wherein the penetrating element has a smaller cross-sectional size than a cross-sectional size of the elongate shaft.

Alternatively or additionally to any of the embodiments above, further comprising one or more steering wires disposed within the elongate shaft.

Alternatively or additionally to any of the embodiments above, wherein the distal end of the elongate shaft includes an angled edge.

Alternatively or additionally to any of the embodiments above, wherein the penetrating element is deflectable.

Alternatively or additionally to any of the embodiments above, wherein the penetrating element is rotatable relative to the elongate shaft.

Alternatively or additionally to any of the embodiments above, wherein the ultrasound transducer is positioned on the penetrating element.

Alternatively or additionally to any of the embodiments above, wherein applying heat to the electrical conductor causes a length of the coiled structure to contract.

Alternatively or additionally to any of the embodiments above, wherein the elongate shaft comprises a plurality of inflatable balloons disposed adjacent the distal end thereof.

Alternatively or additionally to any of the embodiments above, further comprising at least one radiopaque marker.

Alternatively or additionally to any of the embodiments above, wherein the steering wire comprises a strand of polymer fiber twisted with a conductor into a coiled structure.

Alternatively or additionally to any of the embodiments above, wherein the steering wire comprises a plurality of strands of polymer fibers twisted with a conductor into a coiled structure.

Alternatively or additionally to any of the embodiments above, wherein applying heat to the electrical conductor causes a length of the coiled structure to contract.

Alternatively or additionally to any of the embodiments above, wherein applying electricity to the electrical conductor causes a length of the coiled structure to contract.

Alternatively or additionally to any of the embodiments above, wherein the coiled structure comprises a tightly wound coil.

Alternatively or additionally to any of the embodiments above, wherein the coiled structure comprises a loosely wound coil.

Alternatively or additionally to any of the embodiments above, wherein the steering wire comprises a bimetallic strip.

An example biopsy assembly may comprise:

a guide element including a first lumen, a second lumen, and a third lumen;

a catheter including an elongate shaft, the elongate shaft having a proximal end, a distal end and a working lumen extending between the proximal end and the distal end; and

a steering wire disposed within a wall of the elongate shaft;

wherein the elongate shaft tapers from a first outer diameter proximal to the distal end to a second outer diameter smaller than the first outer diameter adjacent to the distal end.

Alternatively or additionally to any of the embodiments above, wherein a diameter of the working lumen of the elongate shaft is constant along a length of the taper from the first outer diameter to the second outer diameter of the elongate shaft.

Alternatively or additionally to any of the embodiments above, wherein the working lumen of the elongate shaft is configured to slidably receive an ultrasound device or a biopsy tool.

Alternatively or additionally to any of the embodiments above, further comprising one or more sensors disposed on an outer surface of the catheter, the one or more sensors configured to provide an X,Y,Z location of the catheter.

Alternatively or additionally to any of the embodiments above, further comprising an elongate member disposed within the working lumen of the elongate shaft, the elongate member including a plurality of anchoring markers.

Alternatively or additionally to any of the embodiments above, further comprising an ultrasound transducer positioned adjacent the distal end of the elongate shaft.

Alternatively or additionally to any of the embodiments above, wherein the elongate shaft comprising an outer tubular member and an inner tubular member, the inner tubular member configured to distally advance and proximally retract relative to the outer tubular member.

Alternatively or additionally to any of the embodiments above, wherein the steering wire comprises a strand of polymer fiber twisted with a conductor into a coiled structure.

Alternatively or additionally to any of the embodiments above, wherein the steering wire comprises a plurality of strands of polymer fibers twisted with a conductor into a coiled structure.

Alternatively or additionally to any of the embodiments above, wherein applying heat to the electrical conductor causes a length of the coiled structure to contract.

Alternatively or additionally to any of the embodiments above, wherein applying electricity to the electrical conductor causes a length of the coiled structure to contract.

Alternatively or additionally to any of the embodiments above, wherein the coiled structure comprises a tightly wound coil.

Alternatively or additionally to any of the embodiments above, wherein the coiled structure comprises a loosely wound coil.

Alternatively or additionally to any of the embodiments above, wherein the steering wire comprises a bimetallic strip.

Alternatively or additionally to any of the embodiments above, further comprising at least one radiopaque marker.

An example method of providing a forward viewing ultrasound image may comprise:

advancing an ultrasound device through a body lumen to a target location, the ultrasound device comprising:
an elongate shaft having a proximal end, a distal end, an inflation lumen, and a vacuum lumen;
an inflatable balloon positioned proximal to the distal end of the elongate shaft; and
an ultrasound transducer positioned adjacent to the distal end of the elongate shaft;

inflating the inflatable balloon;

applying a vacuum to the body lumen distal to the inflatable balloon;

delivering energy to the ultrasound transducer; and

processing an acoustic energy feedback to generate an image.

Alternatively or additionally to any of the embodiments above, further comprising deflating the inflatable balloon.

Alternatively or additionally to any of the embodiments above, further comprising advancing the ultrasound device through the body lumen based on the generated image.

Alternatively or additionally to any of the embodiments above, wherein the inflatable balloon fills a gap between an outer surface of the inflatable balloon and an inner surface of the body lumen.

Alternatively or additionally to any of the embodiments above, wherein delivering energy to the ultrasound transducer comprises supplying electrical energy to the ultrasound transducer to generate acoustic energy.

Alternatively or additionally to any of the embodiments above, wherein the acoustic energy is directed distal to the distal end of the elongate shaft.

Alternatively or additionally to any of the embodiments above, further comprising pumping an infusion fluid into the body lumen while the vacuum is applied.

An example biopsy assembly may comprise:

a guide element including a first lumen, a second lumen, and a third lumen;

a catheter including an elongate shaft, the elongate shaft having a proximal end, a distal end and a lumen extending between the proximal end and the distal end; and

a plurality of inflatable balloons positioned adjacent the distal end of the elongate shaft;

wherein the plurality of inflatable balloons are configured to be inflated and/or deflated independent of one another.

Alternatively or additionally to any of the embodiments above, further comprising at least one radiopaque marker.

An example biopsy assembly may comprise:

a guide element including a first lumen, a second lumen, and a third lumen;

a catheter including an elongate shaft, the elongate shaft comprising an outer tubular member and an inner tubular member, the inner tubular member configured to distally advance and proximally retract relative to the outer tubular member.

Alternatively or additionally to any of the embodiments above, further comprising at least one radiopaque marker.

Alternatively or additionally to any of the embodiments above, wherein an inner surface of the outer tubular member comprises a plurality of grooves.

Alternatively or additionally to any of the embodiments above, wherein an outer surface of the inner tubular member comprises a threaded region.

Alternatively or additionally to any of the embodiments above, wherein the threaded region of the inner tubular member is configured to engage the grooves of the outer tubular member.

Alternatively or additionally to any of the embodiments above, wherein an outer surface of the inner tubular member comprises a plurality of grooves.

Alternatively or additionally to any of the embodiments above, wherein an inner surface of the outer tubular member comprises a threaded region.

Alternatively or additionally to any of the embodiments above, wherein the threaded region of the outer tubular member is configured to engage the grooves of the inner tubular member.

Alternatively or additionally to any of the embodiments above, wherein rotation of one of the inner or outer tubular members in a first direction causes the inner tubular member to distally advance.

Alternatively or additionally to any of the embodiments above, wherein rotation of one of the inner or outer tubular members in a second direction generally opposite the first direction causes the outer tubular member to proximally retract.

In one implementation, a forward viewing ultrasound device may comprise an elongate shaft having a proximal end, a distal end, an inflation lumen, and a vacuum lumen. An inflatable balloon may be positioned proximal to the distal end of the elongate shaft. An ultrasound transducer may be positioned adjacent to the distal end of the elongate shaft. A power and control unit may be in electrical communication with the ultrasound transducer and a vacuum source may be in fluid communication with the vacuum lumen.

In a second implementation, an ultrasound device may comprise an elongate shaft having a proximal end, a distal end, and a lumen extending therethrough. A penetrating element configured to penetrate tissue may be positioned adjacent to the distal end of the elongate shaft. An ultrasound transducer may be positioned proximal to the distal tip and adjacent to the distal end of the elongate shaft. A power and control unit may be in electrical communication with the ultrasound transducer.

In a third implementation, a biopsy assembly may comprise a guide element including a first lumen, a second lumen, and a third lumen. The assembly may further comprise a catheter including an elongate shaft. The elongate shaft may have a proximal end, a distal end, and a working lumen extending between the proximal end and the distal end. A steering wire may be disposed within a wall of the elongate shaft. The elongate shaft may taper from a first outer diameter proximal to the distal end to a second outer diameter smaller than the first outer diameter adjacent to the distal end.

### BRIEF DESCRIPTION OF THE DRAWINGS

The disclosure may be more completely understood in consideration of the following detailed description in connection with the accompanying drawings, in which:

FIG. 1 is a plan view of an example biopsy tool accessing a peripheral lung nodule;

FIG. 2 is a perspective view of a distal portion of an illustrative ultrasound device;

FIG. 3 is a cross-sectional view of the illustrative ultrasound device of FIG. 2, taken at line 3-3 in FIG. 2;

FIGS. 4A-4E illustrate an illustrative method of using the ultrasound device of FIGS. 2 and 3;

FIG. 5 is a side view of a distal portion of another illustrative ultrasound device;

FIG. 6 is a side view of a distal portion of another illustrative ultrasound device;

FIG. 7 is a partial perspective view of a distal portion of another illustrative ultrasound device;

FIG. 8 is a cross-sectional view of a distal portion of another illustrative ultrasound device;

FIG. 9 is a side view of a distal portion of another illustrative ultrasound device;

FIG. 10 is a side view of an illustrative biopsy assembly;

FIG. 11 is a cross-sectional view of a catheter of the illustrative biopsy assembly of FIG. 10;

FIG. 12 is a side view of a catheter of the illustrative biopsy assembly of FIG. 10;

FIG. 13 is a side view of a distal portion of another illustrative catheter;

FIG. 14 is a side view of a distal portion of another illustrative catheter;

FIG. 15 is a side view of a distal portion of another illustrative catheter; and

FIG. 16 is a side view of a distal portion of another illustrative catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar structures in different drawings are numbered the same. The drawings, which are not necessarily to scale, depict illustrative embodiments and are not intended to limit the scope of the disclosure.

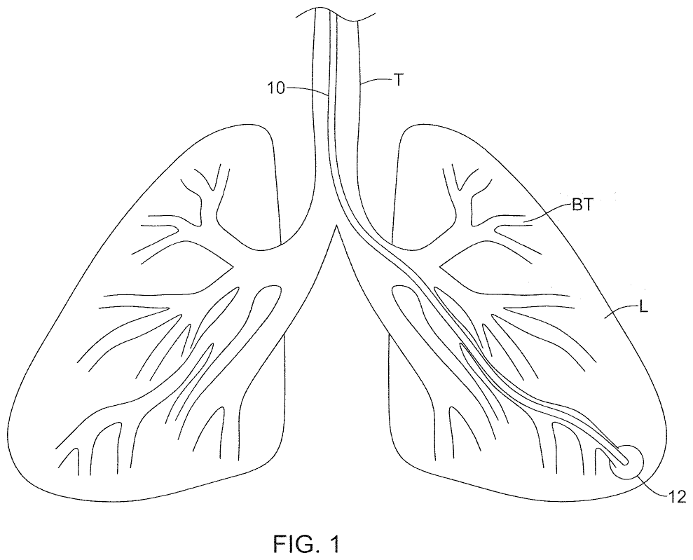

FIG. 1 illustrates a plan view of an example biopsy system 10 advanced through the trachea T and the bronchial tree BT to a peripheral nodule 12 within the lung L. In some instances, the nodule or lesion 12 may be located in a peripheral region of the lung which may be difficult to access and visualize. It may be desirable to aid in the visualization and confirmation of cancerous and/or benign nodules located in the lungs. The global lung cancer epidemic, combined with the adoption of lung cancer screening, may result in an increasing number of suspicious solitary pulmonary nodules (SPNs) found on chest CT scans. Suspicious SPNs, which typically exist in the periphery of the lungs, may be difficult to access and diagnose using current bronchoscopic technologies designed primarily for the central airway. Peripheral lung nodules, or solitary pulmonary nodules (SPNs), may be rounded masses measuring up to 3 centimeters (cm), which can be benign or malignant. When a SPN is identified, it may need to be diagnosed with a biopsy. While the present disclosure is described with respect to lung nodules, it is contemplated that the methods and devices described herein can be applied to other parts of the anatomy, such as, but not limited to gastrointestinal, urological, gynecological, etc.

Some current devices may use ultrasound to help guide pulmonary physicians to a location where a computed tomography (CT) scan revealed the approximate location of a solitary pulmonary nodule. For example, a linear endobronchial ultrasound (EBUS), also known as convex probe EBUS, may image to the side of the device. A radial probe EBUS may image radially 360°. These devices cannot image, or look, forward because the air in the bronchi does not allow for the passage of ultrasound waves. For this reason, these devices can only look to the when the distal end of the probe is against the side of the bronchi lumen. It may be desirable to provide an ultrasound device that has forward imaging capabilities to facilitate the location of suspicious SPNs. In other implementations, example biopsy system 10 may be used for detection in other parts of a patient's body. For example, the biopsy system 10 may be applied to other parts of the anatomy, such as, but not limited to gastrointestinal, urological, gynecological, etc.

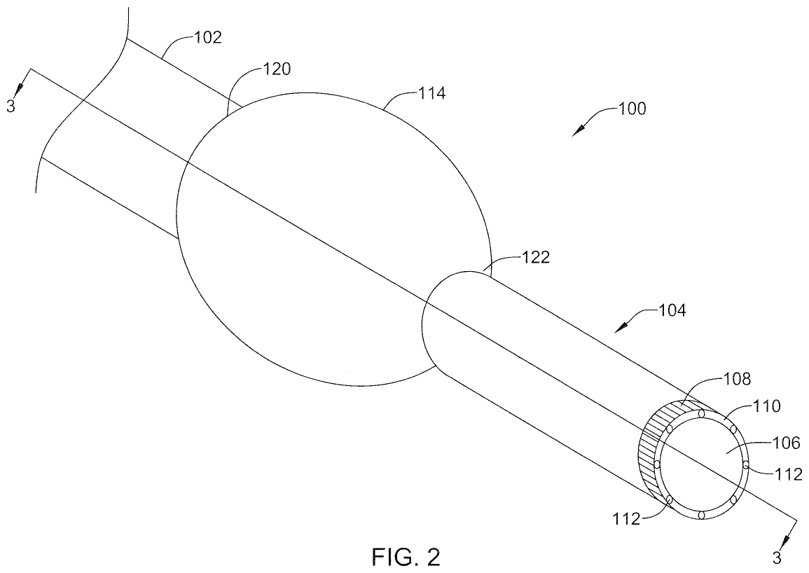

FIG. 2 illustrates a perspective view of a distal portion of an illustrative ultrasound device 100 that may provide forward imaging capabilities. The device 100 may have a long, elongated, flexible tubular shaft 102 that may be inserted into a patient's body for a medical diagnosis/treatment. The elongate shaft 102 may extend proximally from the distal end region 104 to a proximal end configured to remain outside of a patient's body. Although not shown, the proximal end of the elongate shaft 102 may include a hub attached thereto for connecting other treatment devices or providing a port for facilitating other treatments. The elongate shaft 102 may include one or more lumens 106 extending therethrough. In some embodiments, the elongate shaft 102 may include one or more guidewire or auxiliary lumens. While not explicitly shown, the device 100 may include one or more visualization markers, such as radiopaque markers to aid in visibility of the device 100 with the guidance of fluoroscopy imaging.

The device 100 may further include one or more image acquiring portions or ultrasound transducers 108 positioned adjacent the distal end 110 of the elongate shaft 102. While FIG. 2 illustrates a single transducer 108, it is contemplated that the device 100 may include any number of transducers desired, such as, but not limited to, one, two, three, or more. The transducer 108 may be configured to emit and receive acoustic energy (i.e., ultrasound waves) to image portions of the lungs. In some embodiments, the transducer 108 may have a cylindrical shape, however, those skilled in the art will appreciate that any suitable shapes such as, but not limited to, square, rectangular, polygonal, circular, oblong, or the like may also be contemplated. In some instances, such as when a cylindrical transducer is provided, the transducer 108 may extend around the entire circumference of the elongate shaft 102. In an alternative embodiment, however, the transducer 108 may extend partially around the circumference of the elongate shaft 102. For instance, the transducer 108 may include an array of one or more transducers (not shown) positioned about the circumference of the elongate shaft 102. It is further contemplated that the transducer 108 may comprise a plurality of longitudinally spaced transducers.

The transducer 108 may be formed from any suitable material such as, but not limited to, lead zirconate titanate (PZT). It is contemplated that other ceramic or piezoelectric materials may also be used. In some instances, the transducer 108 may include a layer of gold, or other conductive layer, disposed on the acoustically functional areas of the transducer 108 surface for connecting electrical leads to the transducer 108. It is contemplated that the sides/edges of the transducer crystal may be free of conductive material so as not to “short circuit” the transducer 108. In some instances, one or more tie layers may be used to bond the gold to the PZT. For example, a layer of chrome may be disposed between the PZT and the gold to improve adhesion. In other instances, the transducer 108 may include a layer of chrome over the PZT followed by a layer of nickel, and finally a layer of gold. These are just examples. It is contemplated that the layers may be deposited on the PZT using sputter coating, although other deposition techniques may be used as desired.

In some embodiments, an electrical conductor (not explicitly shown), may connect the transducer 108 to a power and control unit configured to remain outside the body. In some embodiments, the electrical conductor(s) may be disposed within a lumen 106 of the elongate shaft 102. In other embodiments, the electrical conductor(s) may extend along an outside surface of the elongate shaft 102. The electrical conductor(s) may provide electricity to the transducer 108, which may then be converted into acoustic energy. It is further contemplated that an additional element may be provided to transmit the acoustic energy received by the transducer 108 to a display or imaging unit to display the ultrasound image.

In order to specifically place or steer ultrasound device 100 to a position adjacent to the intended target, the device 100 may be configured to be deflectable, articulable, or steerable. The elongate shaft 102 may include one or more articulation or deflection mechanism(s) 112 that may allow for the device 100, or portions thereof, to be deflected, articulated, steered, and/or controlled in a desired manner. For example, at least a portion of the elongate shaft 102 may be selectively bent and/or deflected in a desired or predetermined direction. This may, for example, allow a user to orient the device 100 such that the ultrasound transducer 108 is in a desirable position or orientation for navigation to or imaging of a target location.

A variety of deflection mechanisms may be used. In some example embodiments, deflection may be effected by one or more actuation members, such as pull wire(s) 112 extending between a distal portion 104 of the elongate shaft 102 and an actuation mechanism near the proximal end of the elongate shaft 102. As such, the one or more pull wires may extend both proximally and distally of the desired deflection or bending region or point. This allows a user to actuate (e.g., “pull”) one or more of the pull wires to apply a compression and/or deflection force to at least a portion of the shaft 102 and thereby deflect or bend the elongate shaft 102 in a desired manner. In addition, in some cases the one or more wires may be stiff enough so that they can also be used to provide a pushing and/or tensioning force on the shaft 102, for example, to “push” or “straighten” the shaft 102 into a desired position or orientation. While the device is shown as including eight pull wires 112, it is contemplated that the device 100 may include any number of wires desired, such as, but not limited to, one, two, three, four, or more.

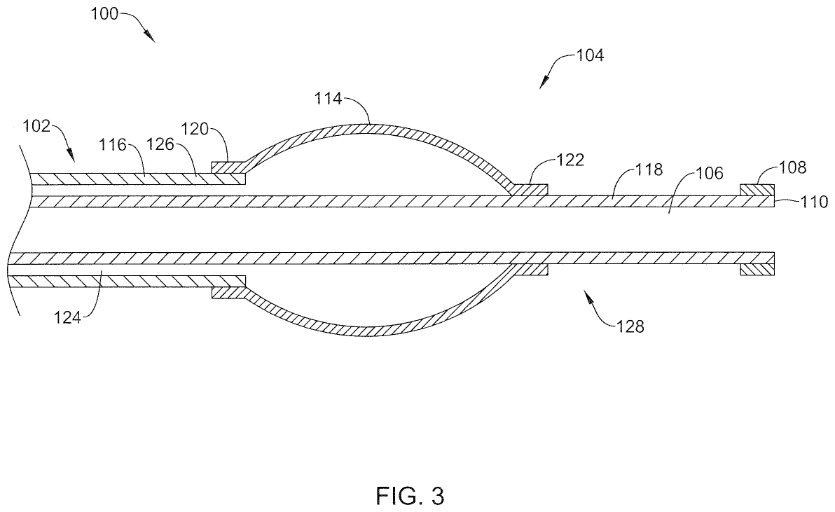

The device 100 may include an inflatable conformable balloon 114 positioned adjacent to the distal end region 104. The balloon 114 may be configured to be inflated within the bronchial tree to create a seal, or fill a gap, within a lumen of the bronchus, as will be discussed in more detail below. Referring additionally to FIG. 3, which illustrates a cross section of device 100 taken at line 3-3 of FIG. 2, the balloon 114 may be secured to the elongate shaft 102 to define an inflation lumen 124. For example, the elongate shaft 102 may include an outer tubular member 116 and an inner tubular member 118 disposed within the lumen of the outer tubular member 116. The balloon 114 may have a proximal end region 120 secured adjacent to a distal end region 126 of the outer tubular member 116 and a distal end region 122 secured adjacent to a distal end region 128 of the inner tubular member 118. The annular region between the outer tubular member 116 and the inner tubular member 118 may define the inflation lumen 124. An inflation fluid, such as, but not limited to saline, may be provided to the interior region of the balloon 114 through the inflation lumen 124 to expand or inflate the balloon 114. The inflation fluid may be evacuated through the inflation lumen 124 when it is desired to collapse the balloon 114. It is contemplated that other configurations of the elongate shaft 102 may be utilized to provide an inflation lumen.

In some embodiments, portions of the elongate shaft 102 may be made more flexible than other portions of the elongate shaft 102. For example, the distal portion 104 or the portion of the elongate shaft 102 between the distal end region 122 of the balloon 114 and the distal end 110 of the elongate shaft 102 may be made more flexible than a proximal portion of the elongate shaft 102. This may facilitate steering or deflection of the distal portion 104. In some instances, the distal portion 104 may be made of a more flexible material. In other instances, the distal portion 104 may include segments, ridges, folds, or other structural features to increase flexibility. These are just examples.

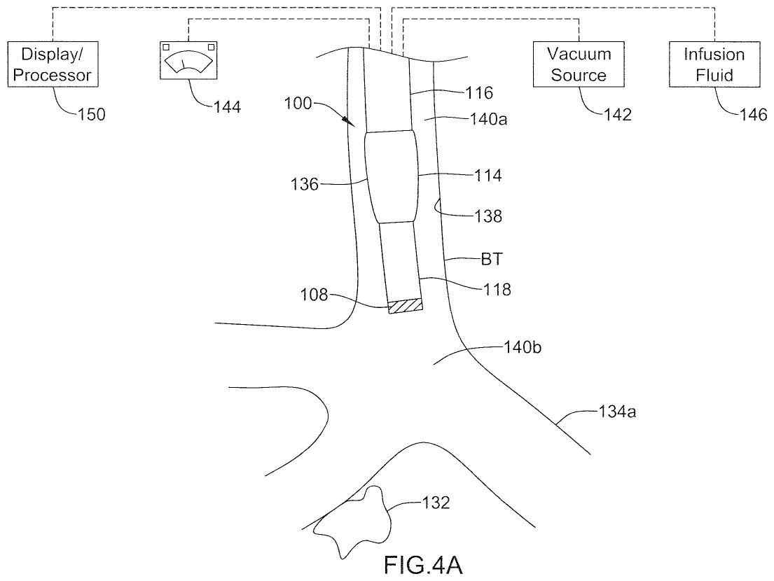

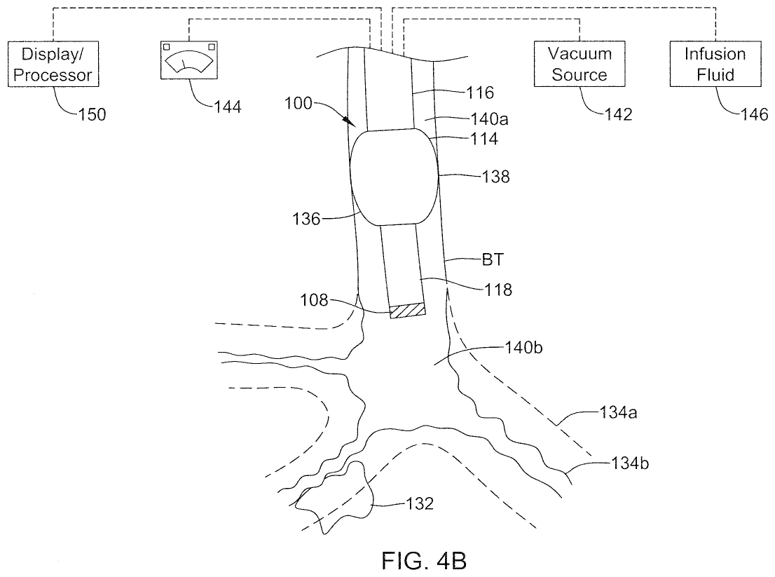

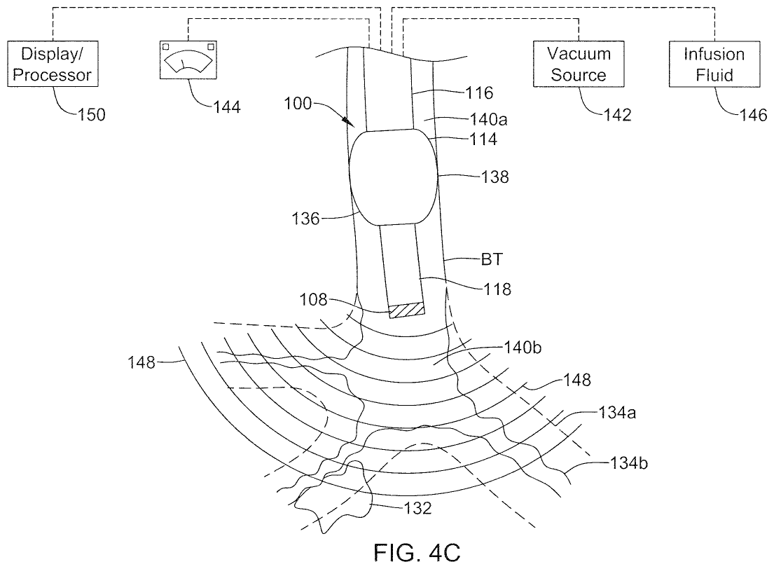

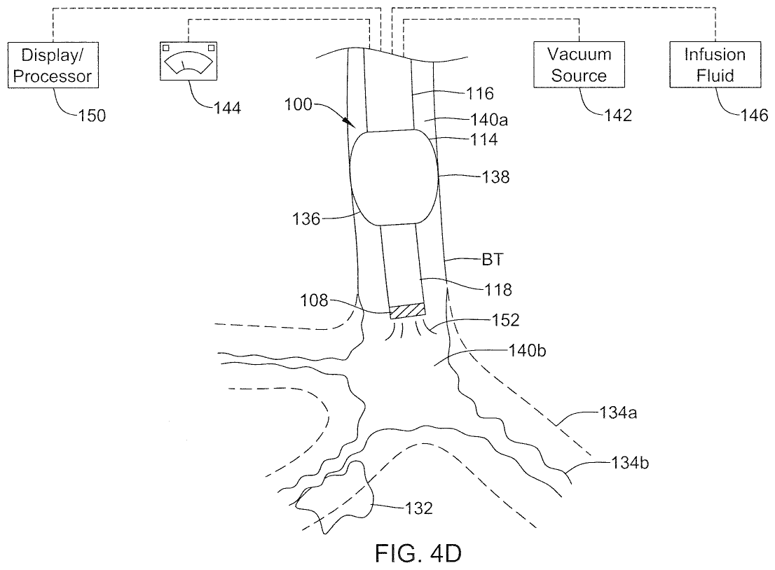

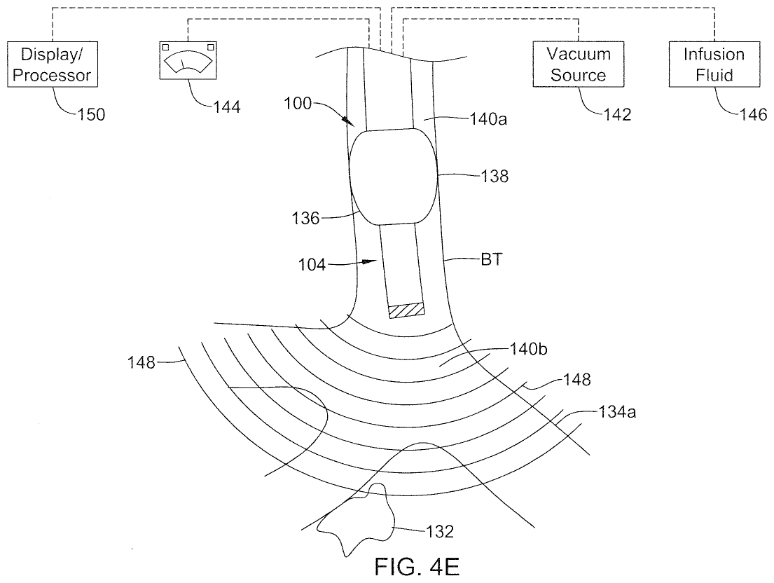

In some instances, the lumen 106 of the device 100 may be configured to be attached to a vacuum pump configured to remain outside the body. In some instances, the lumen 106 may also provide access for other devices, such as, but not limited to, biopsy needles, knifes, etc. When imaging is desired at a particular location, the balloon 114 may be inflated to fill a gap between the balloon 114 and the bronchial lumen. The vacuum pump may then be activated to remove air from the lower level bronchioles (for example, below or distal to the balloon 114), as will be discussed in more detail with respect to FIGS. 4A-4D. Once the air has been evacuated, the ultrasound transducer 108 may be activated to provide an image of the lung distal to and surrounding the distal end 110 of the device 100.

FIGS. 4A-4E illustrate an illustrative method of using ultrasound device 100 to obtain a forward looking image. While the illustrative method is described relative to the lungs, it is contemplated that the ultrasound device 100 may be used in any part of the body or body lumen desired. The ultrasound device 100 may be advanced through the bronchial tree BT with the guidance of fluoroscopy imaging towards the location of the suspicious nodule 132, as shown in FIG. 4A. The location of the nodule 132 may be identified using a CT scan. In some implementations, the ultrasound device 100 may be utilized as a stand-alone device or in combination with a bronchoscope.

Once the distal portion 104 of the device 100 is near, or suspected to be near, the location of the nodule 132, the balloon 114 may be inflated, as shown in FIG. 4B. The outer surface 136 of the balloon 114 may contact the inner surface of the wall 138 of the bronchiole. This may “seal” the airway and effectively prevent air from passing between the bronchioles 140a proximal of the balloon 114 and the bronchioles 140b distal of the balloon 114. Once the balloon 114 has been inflated, the vacuum source 142 may be activated and a vacuum applied to remove air from the bronchiole 140b distal of the balloon 114. In some instances, the walls of the lower bronchial tree BT may slightly collapse or compress under the vacuum from a first configuration 134a to a second collapsed configuration 134b, although this is not required. It is contemplated that removal of the air from the lower bronchial tree may allow acoustic energy to travel distally (or forward) from the distal end 110 of the device. This may allow an image to be generated that looks forward as opposed to the side or radially.

Once the vacuum has been applied, the power and control unit 144 may supply the ultrasound transducer 108 with the necessary energy to generate or emit acoustic waves 148, as shown in FIG. 4C. The ultrasound transducer 108 may also be configured to receive acoustic energy. It is further contemplated that the ultrasound transducer 108 may be in communication with a computer, processor, display or other necessary equipment 150 to process the acoustic energy feedback and/or echoes and generate an image. The generated image may allow a physician to more accurately guide the device 100 to the nodule 132. Once the physician has viewed and/or located the nodule 132, the vacuum may be released and the balloon 114 may be deflated and the device 100 moved towards the nodule 132. The steering mechanism 112 may be actuated to facilitate advancement of the device 100 towards the nodule 132. It is contemplated that if the nodule 132 is not visible on the generated image, the device 100 may need to be repositioned. The physician may release the vacuum, deflate the balloon 114, reposition the device 100, and repeat the steps of inflating the balloon 114, applying a vacuum, and activating the ultrasound transducer 108. Once the device 100 is adjacent to the nodule 132, the vacuum lumen 106 may be used as a working channel to allow for the passage of biopsy tools, needles, knifes, etc. to biopsy or excise the nodule 132. It is further contemplated that a guidewire may be advanced through the lumen 106 to the nodule 132 and used to mark the path to the nodule 132, thus allowing other devices to be guided to the nodule 132.

Alternatively, or additionally, once the vacuum has been applied to the lower bronchioles, liquid saline solution 152, or other suitable infusion fluid, may be pumped down a lumen of the device 100 to fill the bronchioles 140b distal of the balloon 114, as shown in FIG. 4D. In some instances, a separate infusion lumen may be provided separate from the vacuum lumen 106, although this is not required. The infusion lumen may be in fluid communication with an infusion fluid source 146 configured to remain outside the body. The infusion fluid source 146 may be connected to a proximal end of the device 100 in any suitable manner. It is contemplated that infusion of the saline solution 152 may expand the bronchioles from the collapsed configuration 134b to the original configuration 134a, as shown in FIG. 4E.

Once the infusion fluid 152 has been pumped into the bronchioles 140b distal of the balloon 114, the power and control unit 144 may supply the ultrasound transducer 108 with the necessary energy to generate or emit acoustic waves 148. The ultrasound transducer 108 may also be configured to receive acoustic energy. It is further contemplated that the ultrasound transducer 108 may be in communication with a computer, processor, display or other necessary equipment 150 to process the acoustic energy feedback and/or echoes and generate an image. The generated image may allow a physician to more accurately guide the device to the nodule 132. In some instances, the vacuum may be released after the infusion fluid 152 has filled the bronchioles 140b distal of the balloon 114. The infusion fluid 152 may allow may allow acoustic energy to travel distally (or forward) from the distal end 110 of the device. This may allow an image to be generated that looks forward as opposed to the side or radially. It is contemplated that the balloon 114 may be deflated as the device 100 advanced distally with active ultrasound to guide the device 100 to the nodule 132 on live imaging. Once the device 100 is adjacent to the nodule 132, the infusion fluid 152 may be evacuated form the bronchial tree BT and the vacuum lumen 106 may be used as a working channel to allow for the passage of biopsy tools, needles, knifes, etc. to biopsy or excise the nodule 132. It is further contemplated that a guidewire may be advanced through the lumen 106 to the nodule 132 and used to mark the path to the nodule 132, thus allowing other devices to be guided to the nodule 132.

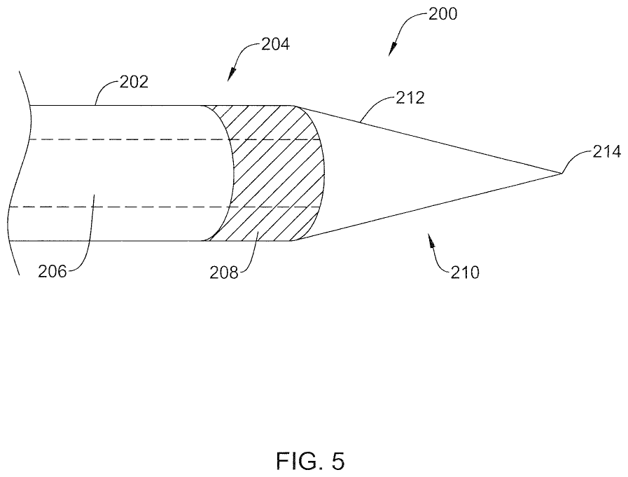

FIG. 5 is a side view of a distal portion of another illustrative ultrasound device 200 that may allow a physician to generate a “forward” image while progressing to a SPN. The device 200 may have a long, elongated, flexible shaft 202 that may be inserted into a patient's body for a medical diagnosis/treatment. The elongate shaft 202 may extend proximally from the distal end region 204 to a proximal end configured to remain outside of a patient's body. Although not shown, the proximal end of the elongate shaft 202 may include a hub attached thereto for connecting other treatment devices or providing a port for facilitating other treatments. The elongate shaft 202 may include one or more lumens 206 extending therethrough, although this is not required. In some instances, the elongate shaft 202 may have a generally solid cross-section. In some embodiments, the elongate shaft 202 may include one or more guidewire or auxiliary lumens. While not explicitly shown, the device 200 may include one or more visualization markers, such as radiopaque markers to aid in visibility of the device 200 with the guidance of fluoroscopy imaging.

The device 200 may further include one or more image acquiring portions or ultrasound transducers 208 positioned adjacent the distal tip 210 of the elongate shall 202. While FIG. 5 illustrates a single transducer 208, it is contemplated that the device 200 may include any number of transducers desired, such as, but not limited to, one, two, three, or more. The transducer 208 may be configured to emit and receive acoustic energy (i.e., ultrasound waves) to image portions of the lungs. In some embodiments, the transducer 208 may have a cylindrical shape, however, those skilled in the art will appreciate that any suitable shapes such as, but not limited to, square, rectangular, polygonal, circular, oblong, or the like may also be contemplated. In some instances, such as when a cylindrical transducer is provided, the transducer 208 may extend around the entire circumference of the elongate shaft 202. In an alternative embodiment, however, the transducer 208 may extend partially around the circumference of the elongate shaft 202. For instance, the transducer 208 may include an array of one or more transducers (not shown) positioned about the circumference of the elongate shaft 202. It is further contemplated that the transducer 208 may comprise a plurality of longitudinally spaced transducers. Those skilled in the art will appreciate that other suitable configurations of the transducer 208 may also be contemplated without departing from the scope and spirit of the present disclosure. The transducer 208 may be similar in form and function to the transducer 108 described above.

In some embodiments, an electrical conductor (not explicitly shown), may connect the transducer 208 to a power and control unit configured to remain outside the body. In some embodiments, the electrical conductor(s) may be disposed within a lumen 206 of the elongate shaft 202. In other embodiments, the electrical conductor(s) may extend along an outside surface of the elongate shaft 202. The electrical conductor(s) may provide electricity to the transducer 208, which may then be converted into acoustic energy. It is further contemplated that an additional element may be provided to transmit the acoustic energy received by the transducer 208 to a display or imaging unit to display the ultrasound image.

In order to specifically place or steer ultrasound device 200 to a position adjacent to the intended target, the device 200 may be configured to be deflectable or articulable or steerable. The elongate shaft 202 may include one or more articulation or deflection mechanism(s) (not explicitly shown) that may allow for the device 200, or portions thereof, to be deflected, articulated, steered and/or controlled in a desired manner. For example, at least a portion of the elongate shaft 202 may be selectively bent and/or deflected in a desired or predetermined direction. This may, for example, allow a user to orient the device 200 such that the ultrasound transducer 208 is in a desirable position or orientation for navigation to or imaging of a target location.

The distal tip 210 of the device 200 may include a penetrating element or needle 212. The needle 212 may include a sharp tip 214 that allows it to relatively easily penetrate into a lumen wall or tissue. In some instances, the tip 214 may have a generally conical shape, although this is not required. Those skilled in the art will appreciate that, other suitable configurations of the tip 214 may also be contemplated without departing from the scope and spirit of the present disclosure.

The device 200 may be advanced through the working channel of an endoscope or bronchoscope through the bronchial tree with the guidance of fluoroscopy imaging towards the location of the suspicious nodule. The location of the nodule may be identified using a CT scan. It is contemplated that the device 200 may be advanced through the bronchial tree without the use of an endoscope. When the device 200 is near, or suspected to be near the nodule, the sharp tip 214 may be pushed into the pulmonary lumen wall, or adjacent tissue. Once the sharp tip 214 has penetrated the wall, a power and control unit may supply the ultrasound transducer 208 with the necessary energy to generate or emit acoustic waves. The ultrasound transducer 208 may also be configured to receive acoustic energy. It is further contemplated that the ultrasound transducer 208 may be in communication with a computer, processor, display or other necessary equipment to process the acoustic energy feedback and/or echoes and generate an image. The generated image may allow a physician to more accurately guide the device 200 to the nodule. It is contemplated that contact with the pulmonary lumen may allow acoustic energy to travel distally (or forward) from the distal tip 210 of the device. This may allow an image to be generated that looks forward as opposed to the side or radially.

Once the physician has viewed and/or located the nodule, the ultrasound energy may be stopped and the device 200 retracted or removed from the pulmonary wall. The physician may then use the generated image to guide the device 200 towards the nodule. It is contemplated that the sharp tip 214 may be caused to penetrate the pulmonary wall and ultrasound images acquired as many times as necessary to guide the device 200 to the nodule. It is contemplated that once the device 200 (and the endoscope) has been positioned adjacent to the nodule, the device 200 may be removed from the endoscope. A biopsy tool, or other device, may then be advanced through the endoscope to obtain a biopsy sample, or perform another procedure. Alternatively, the sharp tip 214 may be equipped with structure, such as a hollow tip, configured to allow the ultrasound device 200 to obtain a biopsy sample.

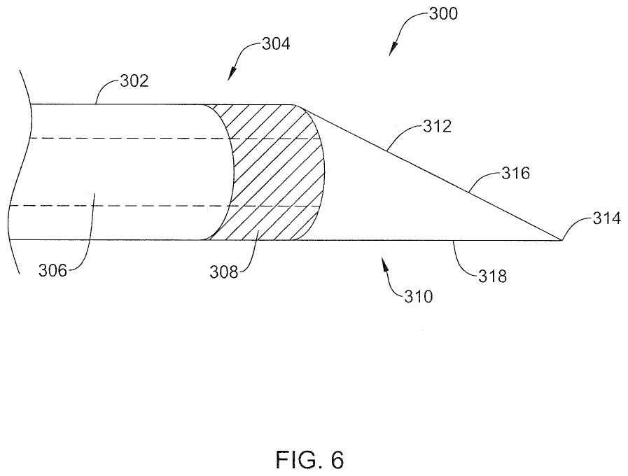

FIG. 6 is a side view of a distal portion of another illustrative ultrasound device 300 that may allow a physician to generate a “forward” image while progressing to a SPN. The device 300 may have a long, elongated, flexible shaft 302 that may be inserted into a patient's body for a medical diagnosis/treatment. The elongate shaft 302 may extend proximally from the distal end region 304 to a proximal end configured to remain outside of a patient's body. Although not shown, the proximal end of the elongate shaft 302 may include a hub attached thereto for connecting other treatment devices or providing a port for facilitating other treatments. The elongate shaft 302 may include one or more lumens 306 extending therethrough, although this is not required. In some instances, the elongate shaft 302 may have a generally solid cross-section. In some embodiments, the elongate shaft 302 may include one or more guidewire or auxiliary lumens. While not explicitly shown, the device 300 may include one or more visualization markers, such as radiopaque markers to aid in visibility of the device 300 with the guidance of fluoroscopy imaging.

The device 300 may further include one or more image acquiring portions or ultrasound transducers 308 positioned adjacent the distal tip 310 of the elongate shaft 302. While FIG. 6 illustrates a single transducer 308, it is contemplated that the device 300 may include any number of transducers desired, such as, but not limited to, one, two, three, or more. The transducer 308 may be configured to emit and receive acoustic energy (i.e., ultrasound waves) to image portions of the lungs. In some embodiments, the transducer 308 may have a cylindrical shape, however, those skilled in the art will appreciate that any suitable shapes such as, but not limited to, square, rectangular, polygonal, circular, oblong, or the like may also be contemplated. In some instances, such as when a cylindrical transducer is provided, the transducer 308 may extend around the entire circumference of the elongate shaft 302. In an alternative embodiment, however, the transducer 308 may extend partially around the circumference of the elongate shaft 302. For instance, the transducer 308 may include an array of one or more transducers (not shown) positioned about the circumference of the elongate shaft 302. It is further contemplated that the transducer 308 may comprise a plurality of longitudinally spaced transducers. Those skilled in the art will appreciate that other suitable configurations of the transducer 308 may also be contemplated without departing from the scope and spirit of the present disclosure. The transducer 308 may be similar in form and function to the transducer 108 described above.

In some embodiments, an electrical conductor (not explicitly shown), may connect the transducer 308 to a power and control unit configured to remain outside the body. In some embodiments, the electrical conductor(s) may be disposed within a lumen 306 of the elongate shaft 302. In other embodiments, the electrical conductor(s) may extend along an outside surface of the elongate shaft 302. The electrical conductor(s) may provide electricity to the transducer 308, which may then be converted into acoustic energy. It is further contemplated that an additional element may be provided to transmit the acoustic energy received by the transducer 308 to a display or imaging unit to display the ultrasound image.

In order to specifically place or steer ultrasound device 300 to apposition adjacent to the intended target, the device 300 may be configured to be deflectable or articulable or steerable. The elongate shaft 302 may include one or more articulation or deflection mechanism(s) (not explicitly shown) that may allow for the device 300, or portions thereof, to be deflected, articulated, steered and/or controlled in a desired manner. For example, at least a portion of the elongate shaft 302 may be selectively bent and/or deflected in a desired or predetermined direction. This may, for example, allow a user to orient the device 300 such that the ultrasound transducer 308 is in a desirable position or orientation for navigation to or imaging of a target location.

The distal tip 310 of the device 300 may include a penetrating element or needle 312. The needle 312 may include a sharp tip 314 that allows it to relatively easily penetrate into a lumen wall or tissue. In some instances, the tip 314 may have a generally angled shape, although this is not required. In some embodiments, the needle 312 may include a first angled side 316 and a second side 318 generally parallel to a longitudinal axis of the elongate shaft 302. It is contemplated that the needle 312 may have a generally flat shape, similar to a knife, scalpel, or lance. For example, the needle 312 may have a thickness smaller than a cross sectional size of the elongate shaft 302 along its entire length. In other instances, the needle 312 may have a cross sectional size and shape that is approximately the same the elongate shaft 302 at a first point and tapers to a smaller cross-sectional size. In some instances, the needle 312 may include features, such as, but not limited to beveled edges or a lumen, to allow the needle 312 to more easily penetrate tissue. Those skilled in the art will appreciate that other suitable configurations of the tip 314 may also be contemplated without departing from the scope and spirit of the present disclosure.

The device 300 may be advanced through the working channel of an endoscope or bronchoscope through the bronchial tree with the guidance of fluoroscopy imaging towards the location of the suspicious nodule. The location of the nodule may be identified using a CT scan. It is contemplated that the device 300 may be advanced through the bronchial tree without the use of an endoscope. When the device 300 is near, or suspected to be near the nodule, the sharp tip 314 may be pushed into the pulmonary lumen wall, or adjacent tissue. Once the sharp tip 314 has penetrated the wall, a power and control unit may supply the ultrasound transducer 308 with the necessary energy to generate or emit acoustic waves. The ultrasound transducer 308 may also be configured to receive acoustic energy. It is further contemplated that the ultrasound transducer 308 may be in communication with a computer, processor, display or other necessary equipment to process the acoustic energy feedback and/or echoes and generate an image. The generated image may allow a physician to more accurately guide the device 300 to the nodule. It is contemplated that contact with the pulmonary lumen may allow acoustic energy to travel distally (or forward) from the distal tip 310 of the device. This may allow an image to be generated that looks forward as opposed to the side or radially.

Once the physician has viewed and/or located the nodule, the ultrasound energy may be stopped and the device 300 retracted or removed from the pulmonary wall. The physician may then use the generated image to guide the device 300 toward the nodule. It is contemplated that the sharp tip 314 may be caused to penetrate the pulmonary wall and ultrasound images acquired as many times as necessary to guide the device 300 to the nodule. It is contemplated that once the device 300 (and the endoscope) has been positioned adjacent to the nodule, the device 300 may be removed from the endoscope. A biopsy tool, or other device, may then be advanced through the endoscope to obtain a biopsy sample, or perform another procedure. Alternatively, the sharp tip 314 may be equipped with structure, such as a hollow tip, configured to allow the ultrasound device 300 to obtain a biopsy sample.

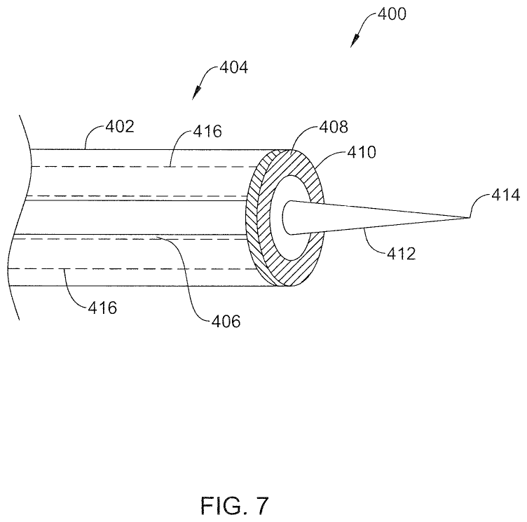

FIG. 7 is a partial perspective view of a distal portion of another illustrative ultrasound device 400 that may allow a physician to generate a “forward” image while progressing to a SPN. The device 400 may have a long, elongated, flexible shaft 402 that may be inserted into a patient's body for a medical diagnosis/treatment. The elongate shaft 402 may extend proximally from the distal end region 404 to a proximal end configured to remain outside of a patient's body. Although not shown, the proximal end of the elongate shaft 402 may include a hub attached thereto for connecting other treatment devices or providing a port for facilitating other treatments. The elongate shaft 402 may include one or more lumens 406 extending therethrough, although this is not required. In some embodiments, the elongate shaft 402 may include one or more guidewire or auxiliary lumens. While not explicitly shown, the device 400 may include one or more visualization markers, such as radiopaque markers to aid in visibility of the device 400 with the guidance of fluoroscopy imaging.

The device 400 may further include one or more image acquiring portions or ultrasound transducers 408 positioned adjacent the distal tip 410 of the elongate shaft 402. While FIG. 7 illustrates a single transducer 408, it is contemplated that the device 400 may include any number of transducers desired, such as, but not limited to, one, two, three, or more. The transducer 408 may be configured to emit and receive acoustic energy (i.e., ultrasound waves) to image portions of the lungs. In some embodiments, the transducer 408 may have a cylindrical shape, however, those skilled in the art will appreciate that any suitable shapes such as, but not limited to, square, rectangular, polygonal, circular, oblong, or the like may also be contemplated. In some instances, such as when a cylindrical transducer is provided, the transducer 408 may extend around the entire circumference of the elongate shaft 402. In an alternative embodiment, however, the transducer 408 may extend partially around the circumference of the elongate shaft 402. For instance, the transducer 408 may include an array of one or more transducers (not shown) positioned about the circumference of the elongate shaft 402. It is further contemplated that the transducer 408 may comprise a plurality of longitudinally spaced transducers. Those skilled in the art will appreciate that other suitable configurations of the transducer 408 may also be contemplated without departing from the scope and spirit of the present disclosure. The transducer 408 may be similar in form and function to the transducer 108 described above.

In some embodiments, an electrical conductor (not explicitly shown), may connect the transducer 408 to a power and control unit configured to remain outside the body. In some embodiments, the electrical conductor(s) may be disposed within a lumen 406 of the elongate shaft 402. In other embodiments, the electrical conductor(s) may extend along an outside surface of the elongate shaft 402. The electrical conductor(s) may provide electricity to the transducer 408, which may then be converted into acoustic energy. It is further contemplated that an additional element may be provided to transmit the acoustic energy received by the transducer 408 to a display or imaging unit to display the ultrasound image.

In order to specifically place or steer ultrasound device 400 to a position adjacent to the intended target, the device 400 may be configured to be deflectable or articulable or steerable. The elongate shaft 402 may include one or more articulation or deflection mechanism(s), such as steering wires or cables 416 that may allow for the device 400, or portions thereof, to be deflected, articulated, steered and/or controlled in a desired manner. For example, at least a portion of the elongate shaft 402 may be selectively bent and/or deflected in a desired or predetermined direction. This may, for example, allow a user to orient the device 400 such that the ultrasound transducer 408 is in a desirable position or orientation for navigation to or imaging of a target location.

The distal tip 410 of the device 400 may include an actuatable penetrating element or an actuatable needle 412. In some instances, the needle 412 may be a biopsy needle. The needle 412 may extend proximally from a sharp or pointed distal tip 414 to a proximal end (not explicitly shown). The proximal end of the needle 412 may affixed to an actuation mechanism in a handle or may otherwise extend to a point where it can be advanced distally or proximally retracted by the physician. It is contemplated that the needle 412 may be slidably disposed within the lumen 406 of the elongate shaft 402 while the device 400 is navigated through the bronchial tree. When so desired, such as, for example, when it is desired to obtain a biopsy sample, the needle 412 may be distally advanced to penetrate the desired tissue. After the sample has been obtained, the needle 412 may be proximally retracted into the lumen 406 to safely withdraw the device 400 from the bronchial tree.

The needle 412 may include a sharp tip 414 that allows it to relatively easily penetrate into a lumen wall or tissue. In some instances, the tip 414 may have a generally conical shape, although this is not required. In some instances, the needle 412 may have an angled or sloped shape. In some embodiments, the needle 412 may include a first angled or sloped side and a second side generally parallel to a longitudinal axis of the elongate shaft 402. It is contemplated that the needle 412 may have a generally flat shape, similar to a knife, scalpel, or lance. For example, the needle 412 may have a thickness smaller than a cross sectional size of the elongate shaft 402 along its entire length. In other instances, the needle 412 may have a cross sectional size and shape that is approximately the same the elongate shaft 402 at a first point and tapers to a smaller cross-sectional size. In some instances, the needle 412 may include features, such as, but not limited to beveled edges or a lumen, to allow the needle 412 to more easily penetrate tissue. It is further contemplated that the needle 412 many include a hollow core or chamber configured to store a tissue sample. Those skilled in the art will appreciate that other suitable configurations of the needle 412 may also be contemplated without departing from the scope and spirit of the present disclosure.

The device 400 may be advanced through the working channel of an endoscope or bronchoscope through the bronchial tree with the guidance of fluoroscopy imaging towards the location of the suspicious nodule. The location of the nodule may be identified using a CT scan. It is contemplated that the device 400 may be advanced through the bronchial tree without the use of an endoscope. When the device 400 is near, or suspected to be near the nodule, the distal tip 410 may be pushed into or brought into contact with the pulmonary lumen wall, or adjacent tissue. It is contemplated that the needle 412 may remain within the lumen 406 or may be distally advanced to penetrate the lumen wall. Once the distal tip 410 contacts the wall, a power and control unit may supply the ultrasound transducer 408 with the necessary energy to generate or emit acoustic waves. The ultrasound transducer 408 may also be configured to receive acoustic energy. It is further contemplated that the ultrasound transducer 408 may be in communication with a computer, processor, display or other necessary equipment to process the acoustic energy feedback and/or echoes and generate an image. The generated image may allow a physician to more accurately guide the device 400 to the nodule. It is contemplated that contact with the pulmonary lumen may allow acoustic energy to travel distally (or forward) from the distal tip 410 of the device. This may allow an image to be generated that looks forward as opposed to the side or radially.

Once the physician has viewed and/or located the nodule, the ultrasound energy may be stopped and the device 400 retracted or removed from the pulmonary wall. The physician may then use the generated image to guide the device 400 toward the nodule. It is contemplated that the distal tip 410 may be caused to contact the pulmonary wall and ultrasound images acquired as many times as necessary to guide the device 400 to the nodule. Once the device 400 has been positioned adjacent to the nodule, the needle 412 may be distally advanced to penetrate the nodule and obtain a tissue sample. It is contemplated that once the device 400 (and the endoscope) has been positioned adjacent to the nodule, the device 400 may be removed from the endoscope. Additional biopsy tools, or other devices, may then be advanced through the endoscope to obtain a biopsy sample, or perform another procedure.

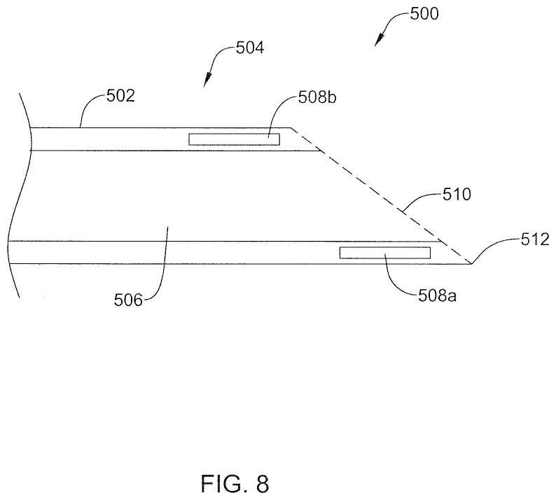

FIG. 8 is a cross-sectional view of a distal portion of another illustrative ultrasound device 500 that may allow a physician to generate a “forward” image while progressing to a SPN. The device 500 may have a long, elongated, flexible shaft 502 that may be inserted into a patient's body for a medical diagnosis/treatment. The elongate shaft 502 may extend proximally from the distal end region 504 to a proximal end configured to remain outside of a patient's body. Although not shown, the proximal end of the elongate shaft 502 may include a hub attached thereto for connecting other treatment devices or providing a port for facilitating other treatments. The elongate shaft 502 may include one or more lumens 506 extending therethrough, although this is not required. In some embodiments, the elongate shaft 502 may include one or more guidewire or auxiliary lumens.

The device 500 may further include one or more image acquiring portions or ultrasound transducers 508a, 508b positioned adjacent the distal tip 510 of the elongate shaft 502. In some instances, the transducers 508a, 508b may be embedded in a wall of the elongate shaft 502 while in other instances, the transducers 508a, 508b may be disposed on an inner and/or an outer surface of the elongate shaft 502. While FIG. 8 illustrates two transducers 508a, 508b, it is contemplated that the device 500 may include any number of transducers desired, such as, but not limited to, one, two, three, or more. The device 500 may include a first transducer 508a configured to emit acoustic energy and a second transducer 508b configured to receive acoustic energy. The reverse configuration is also contemplated. For example, the first transducer 508a may be configured to receive acoustic energy and the second transducer 508b configured to emit acoustic energy. The transducers 508a, 508b may function together to provide an image. In some instances, each transducer 508a, 508b may be configured to both emit and receive acoustic energy. In some embodiments, the transducers 508a, 508b may have a rectangular shape, however, those skilled in the art will appreciate that any suitable shapes such as, but not limited to, square, cylindrical, polygonal, circular, oblong, or the like may also be contemplated. In some instances, the transducers 508a, 508b may extend partially around the circumference of the elongate shaft 502. In an alternative embodiment, however, the transducers 508a, 508b may extend around the entire circumference of the elongate shaft 502. For instance, the transducers 508a, 508b may include an array of one or more transducers (not shown) positioned about the circumference of the elongate shaft 502. It is further contemplated that the transducers 508a, 508b may comprise a plurality of longitudinally spaced transducers. Those skilled in the art will appreciate that other suitable configurations of the transducers 508a, 508b may also be contemplated without departing from the scope and spirit of the present disclosure. The transducer 508a, 508b may be similar in form and function to the transducer 108 described above.

In some embodiments, an electrical conductor (not explicitly shown), may connect the transducers 508a, 508b to a power and control unit configured to remain outside the body. In some instances, separate electrical conductors may be provided to each of the transducers 508a, 508b, although this is not required. In some embodiments, the electrical conductor(s) may be disposed within a lumen 506 of the elongate shaft 502. In other embodiments, the electrical conductor(s) may extend along an outside surface of the elongate shaft 502. The electrical conductor(s) may provide electricity to the transducer 508, which may then be converted into acoustic energy. It is further contemplated that an additional element may be provided to transmit the acoustic energy received by the transducer 508 to a display or imaging unit to display the ultrasound image.

In order to specifically place or steer ultrasound device 500 to a position adjacent to the intended target, the device 500 may be configured to be deflectable or articulable or steerable. The elongate shaft 502 may include one or more articulation or deflection mechanism(s), such as steering wires or cables (not explicitly shown) that may allow for the device 500, or portions thereof, to be deflected, articulated, steered and/or controlled in a desired manner. For example, at least a portion of the elongate shaft 502 may be selectively bent and/or deflected in a desired or predetermined direction. This may, for example, allow a user to orient the device 500 such that the ultrasound transducer 508 is in a desirable position or orientation for navigation to or imaging of a target location.

The distal tip 510 of the device 500 may include an angled or beveled edge to form a penetrating element or sharp tip 512. In some instances, the distal tip 510 may function as biopsy needle. The sharp tip 512 may allow the device to relatively easily penetrate into a lumen wall or tissue. It is contemplated that the distal tip 510 many include a hollow core or chamber configured to store a tissue sample. Those skilled in the art will appreciate that other suitable configurations of the distal tip 510 may also be contemplated without departing from the scope and spirit of the present disclosure.

The device 500 may be advanced through the working channel of an endoscope or bronchoscope through the bronchial tree with the guidance of fluoroscopy imaging towards the location of the suspicious nodule. The location of the nodule may be identified using a CT scan. It is contemplated that the device 500 may be advanced through the bronchial tree without the use of an endoscope. When the device 500 is near, or suspected to be near the nodule, the distal tip 510 may be pushed into or brought into contact with the pulmonary lumen wall, or adjacent tissue. Once the distal tip 510 penetrates or contacts the wall, a power and control unit may supply the ultrasound transducer 508a with the necessary energy to generate or emit acoustic waves. The ultrasound transducer 508b may be configured to receive acoustic energy. It is further contemplated that the ultrasound transducers 508a, 508b may be in communication with a computer, processor, display or other necessary equipment to process the acoustic energy feedback and/or echoes and generate an image. The generated image may allow a physician to more accurately guide the device 500 to the nodule. It is contemplated that contact with the pulmonary lumen may allow acoustic energy to travel distally (or forward) from the distal tip 510 of the device. This may allow an image to be generated that looks forward as opposed to the side or radially.

Once the physician has viewed and/or located the nodule, the ultrasound energy may be stopped and the device 500 retracted or removed from the pulmonary wall. The physician may then use the generated image to guide the device 500 toward the nodule. It is contemplated that the distal tip 510 may be caused to contact the pulmonary wall and ultrasound images acquired as many times as necessary to guide the device 500 to the nodule. Once the device 500 has been positioned adjacent to the nodule, the sharp tip 512 may be distally advanced to penetrate the nodule and obtain a tissue sample. It is contemplated that once the device 500 (and the endoscope) has been positioned adjacent to the nodule, the device 500 may be removed from the endoscope. Additional biopsy tools, or other devices, may then be advanced through the endoscope to obtain a biopsy sample, or perform another procedure.

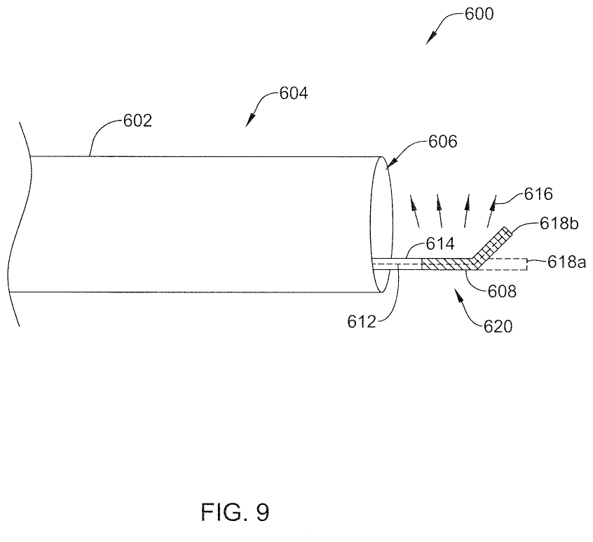

FIG. 9 is a side view of a distal portion of another illustrative ultrasound device 600 that may allow a physician to generate an image while progressing to a SPN. The device 600 may have a long, elongated, flexible shaft 602 that may be inserted into a patient's body for a medical diagnosis/treatment. The elongate shaft 602 may extend proximally from the distal end region 604 to a proximal end configured to remain outside of a patient's body. Although not shown, the proximal end of the elongate shaft 602 may include a hub attached thereto for connecting other treatment devices or providing a port for facilitating other treatments. The elongate shaft 602 may include one or more lumens 606 extending therethrough, although this is not required. In some embodiments, the elongate shaft 602 may include one or more guidewire or auxiliary lumens. While not explicitly shown, the device 600 may include one or more visualization markers, such as radiopaque markers to aid in visibility of the device 600 with the guidance of fluoroscopy imaging.

The device 600 may include a probe 614 slidably disposed within the lumen 606 of the elongate shaft 602. The probe 614 may extend proximally from a distal end region 620 to a proximal end (not explicitly shown). The proximal end of the probe 614 may affixed to an actuation mechanism in a handle or may otherwise extend to a point where it can be advanced distally, proximally retracted, and/or rotated by the physician. The probe 614 may include one or more image acquiring portions or ultrasound transducers 608 positioned adjacent the distal end region 620 of the probe 614. While FIG. 9 illustrates a single transducer 608, it is contemplated that the device 600 may include any number of transducers desired, such as, but not limited to, one, two, three, or more. The transducer 608 may be configured to emit and receive acoustic energy (i.e., ultrasound waves) to image portions of the lungs. In some embodiments, the transducer 608 may have a generally cylindrical shape, however, those skilled in the art will appreciate that any suitable shapes such as, but not limited to, square, rectangular, polygonal, circular, oblong, or the like may also be contemplated. In some instances, the transducer 608 may extend partially around the circumference of the probe 614. In an alternative embodiment, however, the transducer 608 may extend around the entire circumference of the probe 614. For instance, the transducer 608 may include an array of one or more transducers (not shown) positioned about the circumference of the probe 614. It is further contemplated that the transducer 608 may comprise a plurality of longitudinally spaced transducers. Those skilled in the art will appreciate that other suitable configurations of the transducer 608 may also be contemplated without departing from the scope and spirit of the present disclosure. The transducer 608 may be similar in form and function to the transducer 108 described above.

In some embodiments, an electrical conductor (not explicitly shown), may connect the transducer 608 to a power and control unit configured to remain outside the body. In some embodiments, the electrical conductor(s) may be disposed within a lumen of the probe 614. In other embodiments, the electrical conductor(s) may extend along an outside surface of the probe 614. The electrical conductor(s) may provide electricity to the transducer 608, which may then be converted into acoustic energy. It is further contemplated that an additional element may be provided to transmit the acoustic energy received by the transducer 608 to a display or imaging unit to display the ultrasound image.

In some embodiments, the probe 614 may be configured to bend and/or flex. For example, the probe 614 may include one or more steering wires or cables 612 that may allow for the probe 614, or portions thereof, to be deflected, articulated, steered and/or controlled in a desired manner. For example, the probe 614 may be deflectable between a first relatively linear configuration 618a to a second bent configuration 618b. It is contemplated that the second configuration 618b may be at an angle to the longitudinal axis of the probe 614. For example, the angle of the bend may be in the range of greater than 0° and less than 180° from the longitudinal axis of the probe 614. It is further contemplated that the probe 614 may be capable of rotating 360° to allow a user to orient the probe 614 such that the ultrasound transducer 608 is in a desirable position or orientation for navigation to or imaging of a target location.

In order to specifically place or steer ultrasound device 600 to a position adjacent to the intended target, the device 600 may be configured to be deflectable or articulable or steerable. The elongate shaft 602 may include one or more articulation or deflection mechanism(s), such as steering wires or cables (not explicitly shown) that may allow for the device 600, or portions thereof, to be deflected, articulated, steered and/or controlled in a desired manner. For example, at least a portion of the elongate shaft 602 may be selectively bent and/or deflected in a desired or predetermined direction.

The device 600 may be advanced through the working channel of an endoscope or bronchoscope through the bronchial tree with the guidance of fluoroscopy imaging towards the location of the suspicious nodule. The location of the nodule may be identified using a CT scan. It is contemplated that the probe 614 may be capable of being advanced through an endoscope without the elongate shaft 602. It is further contemplated that the device 600 may be advanced through the bronchial tree without the use of an endoscope. When the device 600 is near, or suspected to be near the nodule, the probe 614 and the transducer 608 may be pushed into or brought into contact with the pulmonary lumen wall, or adjacent tissue. It is contemplated that the distal end region 620 of the probe 614 may be deflected and/or the probe 614 rotated as necessary to bring the transducer 608 into contact with tissue. Once the probe 614 and/or transducer 608 penetrates or contacts the wall, a power and control unit may supply the ultrasound transducer 608 with the necessary energy to generate or emit acoustic waves 616. It is further contemplated that the ultrasound transducer 608 may be in communication with a computer, processor, display or other necessary equipment to process the acoustic energy feedback and/or echoes and generate an image. The generated image may allow a physician to more accurately guide the device 600 to the nodule. It is contemplated that contact with the pulmonary lumen may allow acoustic energy to travel distally (or forward) from the distal end region 620 of the device. This may allow an image to be generated that looks forward as opposed to the side or radially.

Once the physician has viewed and/or located the nodule, the ultrasound energy may be stopped and the device 600 retracted or removed from the pulmonary wall. The physician may then use the generated image to guide the device 600 toward the nodule. It is contemplated that the probe 614 and/or transducer 608 may be caused to contact the pulmonary wall and ultrasound to images acquired as many times as necessary to guide the device 600 to the nodule. It is contemplated that once the device 600 (and the endoscope) has been positioned adjacent to the nodule, the device 600 may be removed from the endoscope. A biopsy tool, or other device, may then be advanced through the endoscope to obtain a biopsy sample, or perform another procedure. Alternatively, the probe 614 may be equipped with structure, such as a hollow tip, configured to allow the ultrasound device 600 to obtain a biopsy sample.

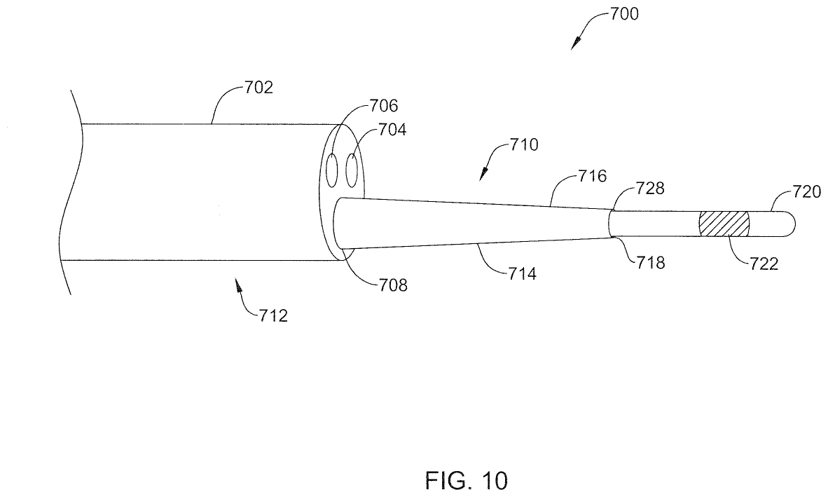

FIG. 10 is a side view of an illustrative biopsy assembly 700 that may be used to obtain a biopsy from a targeted lesion. The assembly 700 may include guide element, endoscope, or bronchoscope 702 and a catheter 710. While not explicitly shown, the assembly 700, or components thereof, may include one or more visualization markers, such as radiopaque markers to aid in visibility of the assembly 700 with the guidance of fluoroscopy imaging. The bronchoscope 702 may extend proximally from a distal end region 712 to a proximal end (not explicitly shown) configured to remain outside the body. Although not shown, the proximal end of the bronchoscope 702 may include a hub attached thereto for connecting other treatment devices or providing a port for facilitating other treatments. The bronchoscope 702 may include a plurality of lumens. For example, the bronchoscope may include a first lumen or a camera lumen 704, a second lumen or a suction/irrigation lumen 706, and a third lumen or a working lumen 708. It is contemplated that the lumens 704, 706, 708 may be arranged in any configuration desired. In some instances, the working lumen 708 may be larger than the camera lumen 704 or the suction/irrigation lumen 706, although this is not required. For example, the working lumen 708 may be sized to slidably receive a catheter having an outer diameter in the range of 1.6-2.0 millimeters (mm) or approximately 1.8 mm. The bronchoscope 702 may have an outer diameter in the range of 5.0-5.5 mm or approximately 5.2 mm.

The catheter 710 may have a long, elongated, flexible shaft 714 that may be inserted into a patient's body for a medical diagnosis/treatment. The elongate shaft 714 may extend proximally from the distal end region 716 to a proximal end configured to remain outside of a patient's body. Although not shown, the proximal end of the elongate shaft 714 may include a hub attached thereto for connecting other treatment devices or providing a port for facilitating other treatments. The elongate shaft 714 may include one or more lumens 718 extending therethrough, although this is not required. In some embodiments, the elongate shaft 714 may include one or more guidewire or auxiliary lumens. In some embodiments, the distal end region 716 of the elongate shaft 714 may be tapered. The catheter 710 may be slidably disposed within the lumen 708 such that the catheter 710 can be distally advanced beyond a distal end of the bronchoscope 702 or proximally retracted to be fully disposed within the lumen 708.

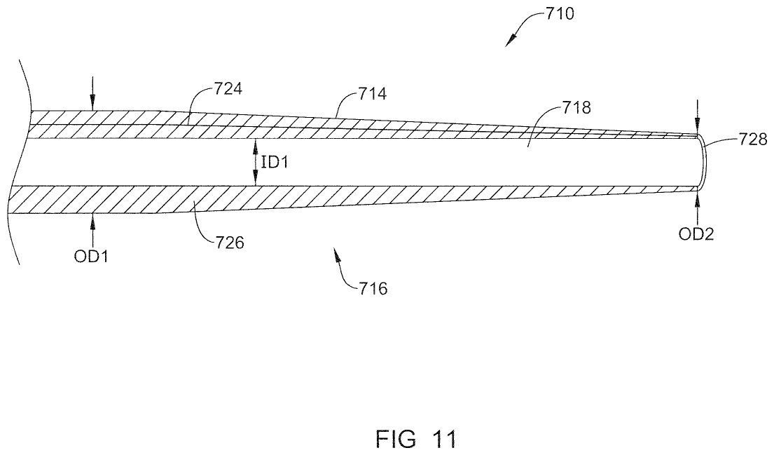

Referring briefly to FIG. 11, which illustrates a cross-sectional view of the distal end region 716 of the elongate shaft 714, the elongate shaft 714 may taper towards the distal end 728 from a first outer diameter OD1 to a second smaller outer diameter OD2. The first outer diameter OD1 may be in the range of 1.6-2.0 mm or approximately 1.8 mm. The second outer diameter OD2 may be in the range of 0.8-1.2 mm or approximately 1.0 mm. In some instances, the thickness of the wall 726 of the elongate shaft 714 may become thinner towards the distal end 728 of the catheter 710 while the inner diameter ID1 remains constant, as shown in FIG. 11. The inner diameter ID1 may be in the range of 0.7-1.1 mm or approximately 0.9 mm. In other embodiments, the inner diameter ID1 may become smaller towards the distal end 728 of the catheter 710. It is contemplated that the tapered outer diameter may allow the catheter 710 to reach the terminal bronchioles of the lungs.

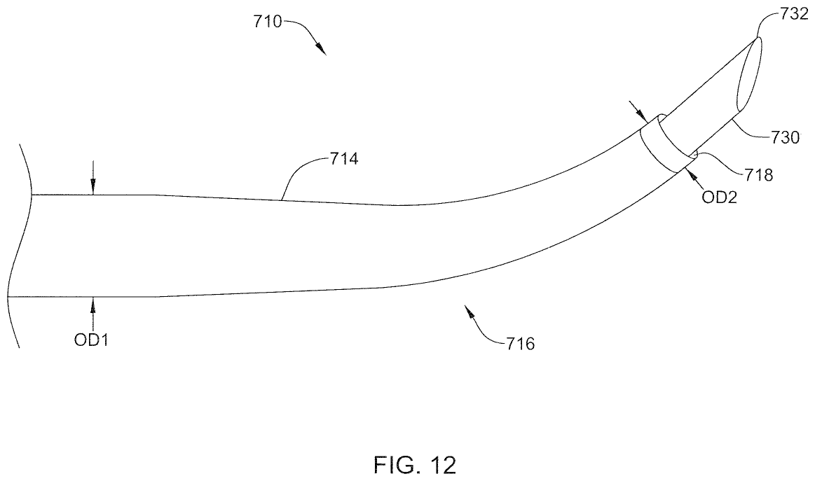

In order to specifically place or steer the catheter 710 to a position adjacent to the intended target, the catheter 710 may be configured to be deflectable or articulable or steerable. The elongate shaft 714 may include one or more articulation or deflection mechanism(s), such as pull wire 724, within a channel in the catheter wall 726 that may allow for the catheter 710 or portions thereof, to be deflected, articulated, steered and/or controlled in a desired manner. For example, at least a portion of the elongate shaft 714 may be selectively bent and/or deflected in a desired or predetermined direction, as shown in FIG. 12. This may, for example, allow a user to orient the catheter 710 such that an ultrasound transducer 722 is in a desirable position or orientation for navigation to or imaging of a target location or a biopsy needle 730 is in a desirable position to obtain a biopsy sample. While the catheter 710 is illustrated as including a single pull wire 724, it is contemplated that the elongate shaft 714 may include one, two, three, four, or more channels containing a pull wire running through its length. The wire 724 could be made of polymers such as nylon, polyester or a metal or metal alloy such as nitinol and or stainless steel. These are just examples. The pull wire 724 can be pulled at the proximal end to steer the distal end region 716 of the catheter 710 around tortuous bends.

The steerability may also be achieved by polymer strands which are anchored to near the distal end 728 and may extend proximally the full length of the catheter 710 to the user interface. Single or multiple strands of polymer fibers could be twisted and paired with a conductor and formed into coiled structure. Applying heat or electricity to the conductor may cause the length of the coil to contract which may in turn deflect the distal end region 716 tip of the catheter 710. Twisting the fibers into tight coils may multiply the contraction effect. Additionally or alternatively, bimetallic strips can be used in the elongate shaft 714 to cause mechanical displacement of the distal end region 716 via temperature change of the strips. The displacement may be dependent on the difference between the thermal coefficients of expansion of the materials. It is contemplated that these types of steering wires may be used with any of the devices described herein.

Returning to FIG. 10, the lumen 718 of the elongate shaft 714 may be sized to receive an intravascular ultrasound (IVUS) or an endobronchial ultrasound (EBUS) probe, such as probe 720 or a biopsy device, such as biopsy needle 730 illustrated in FIG. 12. The probe 720 may be slidably disposed within the lumen 718 such that the probe 720 can be distally advanced beyond a distal end of the elongate shaft 714 or proximally retracted to be fully disposed within the lumen 718. The probe 720 may include an ultrasound transducer 722 configured to provide an image to a physician. In some instances, the probe 720 may be similar in form and function to the ultrasound devices disclosed herein. In other embodiments, the probe 720 may be a linear EBUS, also known as convex probe EBUS, a radial probe EBUS, or other known IVUS device. The probe 720 may provide an image to the physician to help guide the assembly 700 to a desired biopsy region or suspicious nodule.

The physician may guide the assembly 700 through the bronchial tree with the guidance of fluoroscopy imaging towards the location of the suspicious nodule. The location of the nodule may be identified using a CT scan. It is contemplated that, in some instances, catheter 710 may be guided through the bronchial tree without the bronchoscope 702. During navigation of the assembly 700, the probe 720 may be periodically advanced distally beyond the distal end 728 of the catheter 710, as shown in FIG. 10, to obtain an image. The generated image may allow a physician to more accurately guide the assembly 700 to the nodule or target biopsy region. Once the physician has viewed and/or located the nodule, the ultrasound energy may be stopped and the probe 720 retracted proximally into the catheter lumen 718. The physician may then use the generated image to guide the assembly 700 to the nodule. It is contemplated that the probe 720 may be used to generate as many images as necessary to guide the assembly 700 to the nodule.

Once the assembly 700 is positioned adjacent to the nodule, the probe 720 may be completely withdrawn from the catheter lumen 718. A biopsy tool 730 may be distally advanced through the catheter lumen 718, as shown in FIG. 12. While the bronchoscope 702 is not illustrated in FIG. 12, it is contemplated that the bronchoscope 702 may remain in place or adjacent to the nodule, while the probe 720 is replaced with the biopsy tool 730. The biopsy tool 730 may include an angled or beveled edge to form a sharp tip 732. In some instances, the biopsy tool 730 may be a biopsy needle, although this is not required. The sharp tip 732 may allow the biopsy tool 730 to relatively easily penetrate into a lumen wall or tissue. It is contemplated that the biopsy tool 730 many include a hollow core or chamber configured to store a tissue sample. Those skilled in the art will appreciate that other suitable configurations of the biopsy tool 730, or other biopsy devices, may also be contemplated without departing from the scope and spirit of the present disclosure. The biopsy tool 730 may be advanced distally beyond the distal end 728 of the catheter 710 to penetrate the suspect nodule or desired biopsy region and obtain a tissue sample.

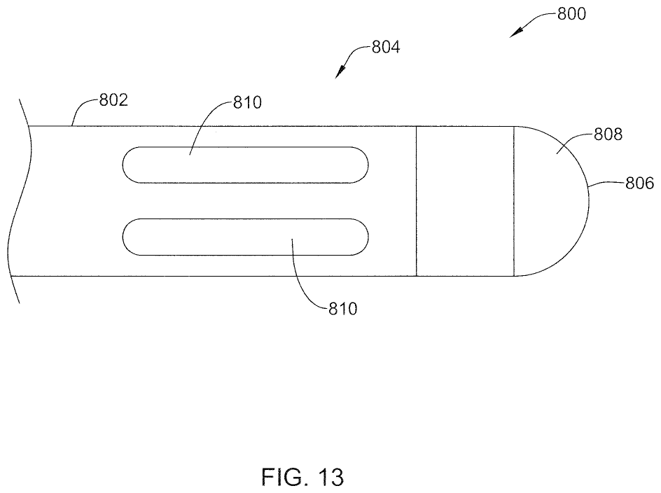

FIG. 13 illustrates a side view of a distal portion of another illustrative catheter 800 that may be used in combination with a biopsy assembly, such as assembly 700. The catheter 800 may have a long, elongated, flexible shaft 802 that may be inserted into a patient's body for a medical diagnosis/treatment. The elongate shaft 802 may extend proximally from the distal end region 804 to a proximal end configured to remain outside of a patient's body. Although not shown, the proximal end of the elongate shaft 802 may include a hub attached thereto for connecting other treatment devices or providing a port for facilitating other treatments. The elongate shaft 802 may include one or more lumens (not explicitly shown) extending therethrough, although this is not required. In some embodiments, the elongate shaft 802 may include one or more guidewire or auxiliary lumens. While not explicitly shown, the catheter 800 may include one or more visualization markers, such as radiopaque markers to aid in visibility of the catheter 800 with the guidance of fluoroscopy imaging.

In order to specifically place or steer the catheter 800 to a position adjacent to the intended target, the catheter 800 may be configured to be deflectable or articulable or steerable. The elongate shaft 802 may include one or more articulation or deflection mechanism(s), such as steering wires or cables (not explicitly shown) that may allow for the catheter 800, or portions thereof, to be deflected, articulated, steered and/or controlled in a desired manner. For example, at least a portion of the elongate shaft 802 may be selectively bent and/or deflected in a desired or predetermined direction. This may, for example, allow a user to orient the catheter 800 in a desirable position or orientation for navigation to a target location.

The distal tip 806 of the catheter 800 may be an echogenic ultrasound tip to provide visibility of the catheter 800. For example, the distal tip 806 may comprise an ultrasound transducer 808. The transducer 808 may be configured to emit and receive acoustic energy (i.e., ultrasound waves) to image portions of the lungs. In some embodiments, the ultrasound transducer 808 may have a hemispherical shape, although this is not required. Those skilled in the art will appreciate that other suitable configurations of the transducer 808 may also be contemplated without departing from the scope and spirit of the present disclosure. The transducer 808 may be similar in form and function to the transducers described above, such as transducer 108.

In some embodiments, an electrical conductor (not explicitly shown), may connect the transducer 808 to a power and control unit configured to remain outside the body. In some embodiments, the electrical conductor(s) may be disposed within a lumen of the elongate shaft 802. In other embodiments, the electrical conductor(s) may extend along an outside surface of the elongate shaft 802. The electrical conductor(s) may provide electricity to the transducer 808, which may then be converted into acoustic energy. It is further contemplated that an additional element may be provided to transmit the acoustic energy received by the transducer 808 to a display or imaging unit to display the ultrasound image.

The catheter 800 may further include one or more sensors 810 configured to emit a signal to provide an X, Y, Z location (using the Cartesian coordinate system) of the catheter 800. The sensors 810 may be calibrated to provide a known origin. In some instances, the sensors 810 may be able to sense the nodule. In some instances, when administered into the body, some molecules, such as but not limited to, certain nanoparticles, may accumulate in tumorous tissue much more than normal tissue. The underlying mechanism for their entrapment is known as the Enhanced Permeability and Retention (EPR) effect. The EPR effect of certain molecules may allow a marking agent, such as but not limited to a fluorescent marker, methylene blue, gold nanoparticles, a paramagnetic nanoparticle or quantum dots (silicon), to accumulate in a lesion. If the nodule has been marked with such a marking agent, the sensors 810 may be able to sense the marking agents which act as a beacon for the sensors 810.

The device 800 may be advanced through the working channel of an endoscope or bronchoscope through the bronchial tree with the guidance of fluoroscopy imaging towards the location of the suspicious nodule. The location of the nodule may be identified using a CT scan. It is contemplated that the device 800 may be advanced through the bronchial tree without the use of an endoscope. When the device 800 is near, or suspected to be near the nodule, the distal tip 806 may be pushed into or brought into contact with the pulmonary lumen wall, or adjacent tissue. Once the distal tip 806 contacts the wall, a power and control unit may supply the ultrasound transducer 808 with the necessary energy to generate or emit acoustic waves. The ultrasound transducer 808 may also be configured to receive acoustic energy. It is further contemplated that the ultrasound transducer 808 may be in communication with a computer, processor, display or other necessary equipment to process the acoustic energy feedback and/or echoes and generate an image. The generated image, in addition to the X,Y,Z coordinate information provided by the sensors 810, may allow a physician to more accurately guide the catheter 800 to the nodule. It is contemplated that contact with the pulmonary lumen may allow acoustic energy to travel distally (or forward) from the distal tip 810 of the device. This may allow an image to be generated that looks forward as opposed to the side or radially.

Once the physician has viewed and/or located the nodule, the ultrasound energy may be stopped and the catheter 800 retracted or removed from the pulmonary wall. The physician may then use the generated image and/or the X,Y,Z coordinate information provided by the sensors 810 to guide the catheter 800 to the nodule. It is contemplated that the distal tip 810 may be caused to contact the pulmonary wall and ultrasound images acquired as many times as necessary to guide the catheter 800 to the nodule. Once the catheter 800 has been positioned adjacent to the nodule, a guidewire may be advanced through the bronchoscope to mark the position of the nodule. Additional biopsy tools, or other devices, may then be advanced over the guidewire or through the bronchoscope to obtain a biopsy sample, or perform another procedure. In other embodiments, the distal tip 806 of the catheter may be configured to allow a biopsy tool to be advanced through a lumen of the catheter to obtain the biopsy sample.

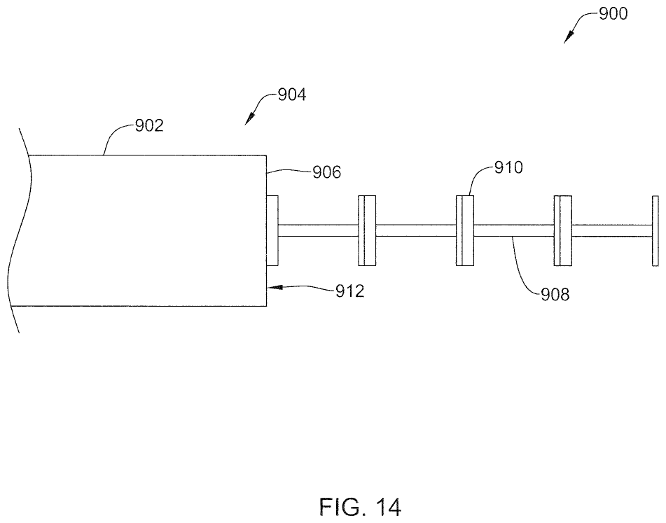

FIG. 14 illustrates a side view of a distal portion of another illustrative catheter 900 that may be used in combination with a biopsy assembly, such as assembly 700. The catheter 900 may have a long, elongated, flexible shaft 902 that may be inserted into a patient's body for a medical diagnosis/treatment. The elongate shaft 902 may extend proximally from the distal end region 904 to a proximal end configured to remain outside of a patient's body. Although not shown, the proximal end of the elongate shaft 902 may include a hub attached thereto for connecting other treatment devices or providing a port for facilitating other treatments. The elongate shaft 902 may include one or more lumens 912 extending therethrough, although this is not required. In some embodiments, the elongate shaft 902 may include one or more guidewire or auxiliary lumens. While not explicitly shown, the catheter 900 may include one or more visualization markers, such as radiopaque markers to aid in visibility of the catheter 900 with the guidance of fluoroscopy imaging.

In order to specifically place or steer the catheter 900 to a position adjacent to the intended target, the catheter 900 may be configured to be deflectable or articulable or steerable. The elongate shaft 902 may include one or more articulation or deflection mechanism(s), such as steering wires or cables (not explicitly shown) that may allow for the catheter 900, or portions thereof, to be deflected, articulated, steered and/or controlled in a desired manner. For example, at least a portion of the elongate shaft 902 may be selectively bent and/or deflected in a desired or predetermined direction. This may, for example, allow a user to orient the catheter 900 in a desirable position or orientation for navigation to a target location.

The catheter 900 may include an elongate member 908 including a plurality of tracking markers with an anchoring means 910 such as T-tags or other fiducial marker. The tracking markers 910 may be made of radiopaque materials that can be seen under fluoroscopy. The elongate member 908 may be slidably disposed within the lumen 912 of the catheter 900 such that can be advanced distally beyond a distal end 906 of the catheter 900 or proximally retracted by the physician. The tracking markers 910 may be secured at the site of the nodule and/or along the path to the nodule. This may provide a marker for the physician to return to at a later time. For example, the tracking markers 910 may be secured at the site where a biopsy sample was obtained. If the nodule is determined to be malignant and needs to be excised, or further procedures are required, the tracking markers 910 may serve as a guide to direct the physician to the biopsy site. It is contemplated that tracking markers with anchoring means may be used in combination with any of the devices disclosed herein to facilitate future procedures.

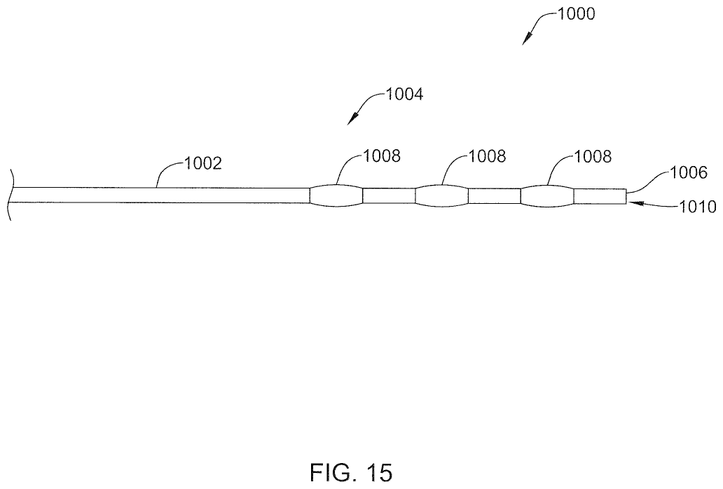

FIG. 15 illustrates a side view of a distal portion of another illustrative catheter 1000 that may be used in combination with a biopsy assembly, such as assembly 700. The catheter 1000 may have a long, elongated, flexible shaft 1002 that may be inserted into a patient's body for a medical diagnosis/treatment. The elongate shaft 1002 may extend proximally from the distal end region 1004 to a proximal end configured to remain outside of a patient's body. Although not shown, the proximal end of the elongate shaft 1002 may include a hub attached thereto for connecting other treatment devices or providing a port for facilitating other treatments. The elongate shaft 1002 may include one or more lumens 1010 extending therethrough, although this is not required. In some embodiments, the elongate shaft 1002 may include one or more guidewire or auxiliary lumens. While not explicitly shown, the catheter 1000 may include one or more visualization markers, such as radiopaque markers to aid in visibility of the catheter 1000 with the guidance of fluoroscopy imaging.

In order to specifically place or steer the catheter 1000 to a position adjacent to the intended target, the catheter 1000 may be configured to be deflectable or articulable or steerable. The elongate shaft 1002 may include one or more articulation or deflection mechanism(s), such as steering wires or cables (not explicitly shown) that may allow for the catheter 1000, or portions thereof, to be deflected, articulated, steered and/or controlled in a desired manner. For example, at least a portion of the elongate shaft 1002 may be selectively bent and/or deflected in a desired or predetermined direction. This may, for example, allow a user to orient the catheter 1000 in a desirable position or orientation for navigation to a target location.

The catheter 1000 may include one or more inflatable balloons 1008. In instances, the elongate shaft 1002 may be formed of two or more coaxially disposed tubular members. This may allow for a central working lumen and separate inflation lumens for inflating the one or more inflatable balloons 1008. The catheter 1000 may be advanced through the working channel of an endoscope or bronchoscope through the bronchial tree with the guidance of fluoroscopy imaging towards the location of the suspicious nodule. The location of the nodule may be identified using a CT scan. It is contemplated that the catheter 1000 may be advanced through the bronchial tree without the use of an endoscope. Visualization techniques, such as ultrasound imaging, or other tracking means may be utilized to facilitate advancement of the catheter 1000 to the desired biopsy location. For example, an ultrasound probe may be disposed within the lumen to provide visual guidance. Once the catheter 1000 is positioned adjacent to the desired biopsy region, one or more of the balloons 1008 may be inflated to secure the catheter 1000. This may allow the physician to remove the ultrasound probe, or other device and load a biopsy tool for performing a biopsy without losing the position of the distal end 1006 of the catheter 1000. In some embodiments, the one or more balloons 1008 may be sequentially inflated and/or deflated to advance or retract the catheter 1000 in incremental movements. For example, sequentially inflation and/or deflation of the balloons 1008 may push or pull the catheter 1000 over short distances.

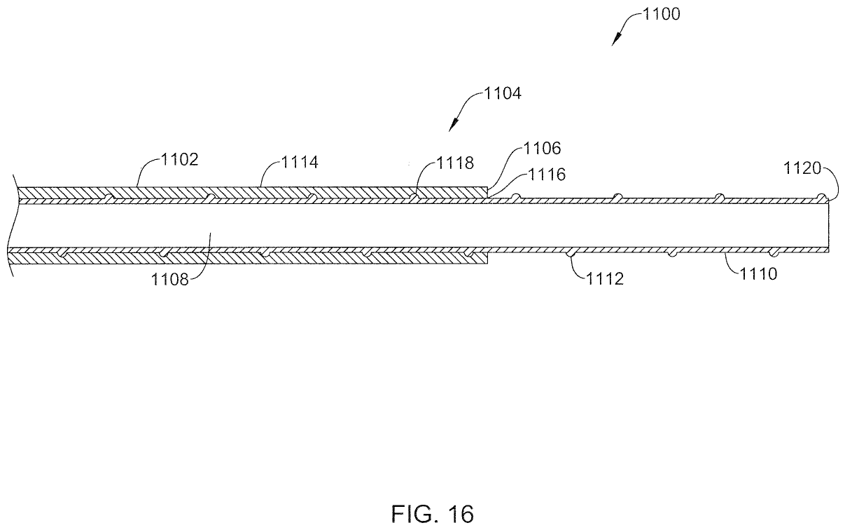

FIG. 16 illustrates a cross-sectional view of a distal portion of another illustrative catheter 1100 that may be used in combination with a biopsy assembly, such as assembly 700. The catheter 1100 may have a long, elongated, flexible shaft 1102 that may be inserted into a patient's body for a medical diagnosis/treatment. The elongate shaft 1102 may extend proximally from the distal end region 1104 to a proximal end configured to remain outside of a patient's body. Although not shown, the proximal end of the elongate shaft 1102 may include a hub attached thereto for connecting other treatment devices or providing a port for facilitating other treatments. The elongate shaft 1102 may include one or more lumens 1108 extending therethrough, although this is not required. The lumen may be configured to receive an ultrasound probe and/or a biopsy tool. In some embodiments, the elongate shaft 1102 may include one or more guidewire or auxiliary lumens. While not explicitly shown, the catheter 1100 may include one or more visualization markers, such as radiopaque markers to aid in visibility of the catheter 1100 with the guidance of fluoroscopy imaging.

In order to specifically place or steer the catheter 1100 to a position adjacent to the intended target, the catheter 1100 may be configured to be deflectable or articulable or steerable. The elongate shaft 1102 may include one or more articulation or deflection mechanism(s), such as steering wires or cables (not explicitly shown) that may allow for the catheter 1100, or portions thereof, to be deflected, articulated, steered and/or controlled in a desired manner. For example, at least a portion of the elongate shaft 1102 may be selectively bent and/or deflected in a desired or predetermined direction. This may, for example, allow a user to orient the catheter 1100 in a desirable position or orientation for navigation to a target location.

The elongate shaft 1102 may include an outer tubular member 1114 and an inner tubular member 1110 movably disposed within a lumen 1116 of the outer tubular member 1114. The outer tubular member 1114 may extend proximally from a distal end 1106 to a proximal end configured to remain outside of a patient's body. Similarly, the inner tubular member 1110 may extend proximally from a distal end 1120 to a proximal end configured to remain outside of a patient's body. In some instances, the inner tubular member 1110 may have any outer diameter similar to an inner diameter of the outer tubular member 1114, although this is not required. In some instances, the outer diameter of the inner tubular member 1110 may be smaller than an inner diameter of the outer tubular member 1114. The inner tubular member 1110 may be configured to be advanced distally beyond a distal end 1106 of the outer tubular member 1114. In some embodiments, the inner tubular member 1110 may include a threaded region 1112 configured to engage corresponding grooves 1118 on the outer tubular member 1114. The reverse configuration is also contemplated. For example, the outer tubular member 1114 may be provided with a threaded region while the inner tubular member 1110 may be provided with corresponding grooves. Clockwise rotation of one of the outer tubular member 1110 or the inner tubular member 1114 may cause the inner tubular member 1114 to advance distally beyond the distal end 1106 of the outer tubular member 1110. Counter-clockwise rotation of one of the outer tubular member 1110 or the inner tubular member 1114 may cause the inner tubular member 1114 to retract proximally within the outer tubular member 1110. The reverse configuration is also contemplated. For example, counter-clockwise rotation may result in distal movement and clockwise rotation may result in proximal movement of the inner tubular member 1114. This telescoping mechanism may be used to extend the reach of the catheter 1100 at limited points of access. For example, the inner tubular member 1114 may be distally advanced, if needed, to access a site that is otherwise inaccessible by the larger outer tubular member 1110.

The materials that can be used for the various components of the delivery devices, and the various members disclosed herein may include those commonly associated with medical devices. For simplicity purposes, the following discussion makes reference to the delivery devices and components of thereof. However, this is not intended to limit the devices and methods described herein, as the discussion may be applied to other similar delivery systems and/or components of delivery systems or devices disclosed herein.

The delivery devices and/or other components of delivery system may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the polymer can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®)), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of the delivery devices and/or other components of delivery system may be doped with, made of, or otherwise include a radiopaque material. Radiopaque materials are understood to be materials capable of producing a relatively bright image on a fluoroscopy screen or another imaging technique during a medical procedure. This relatively bright image aids the user of the delivery devices in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the delivery devices to achieve the same result.

In some embodiments, a degree of Magnetic Resonance Imaging (MRI) compatibility is imparted into the delivery devices. For example, the delivery devices, or portions or components thereof, may be made of a material that does not substantially distort the image and create substantial artifacts (i.e., gaps in the image). Certain ferromagnetic materials, for example, may not be suitable because they may create artifacts in an MRI image. The delivery devices, or portions thereof, may also include and/or be made from a material that the MRI machine can image. Some materials that exhibit these characteristics include, for example, tungsten, cobalt-chromium-molybdenum alloys (e.g., UPN: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

1. An ultrasound device, the ultrasound device comprising:
an elongate shaft having a proximal end, a distal end, and a lumen extending therethrough;
a penetrating element positioned adjacent to the distal end of the elongate shaft, the penetrating element configured to penetrate tissue; and
an ultrasound transducer positioned proximal to a distal tip and adjacent to the distal end of the elongate shaft.

2. The ultrasound device of claim 1, wherein the elongate shaft transitions from a first outer diameter to a second outer diameter.

3. The ultrasound device of claim 2, wherein an inside diameter of the elongate shaft remains constant when the elongate shaft transitions from the first outer diameter to the second outer diameter.

4. The ultrasound device of claim 3, wherein the inside diameter is between 0.7 and 1.1 millimeters and the outside diameter is between 1.6 and 2 millimeters.

5. The ultrasound device of claim 1, wherein the elongate shaft transitions from a first wall thickness to a second wall thickness.

6. The ultrasound device of claim 1, wherein the elongate shaft includes two or more lumens.

7. The ultrasound device of claim 1, wherein the elongate shaft includes one or more visualization markers.

8. The ultrasound device of claim 1, wherein the ultrasound transducer comprises a convex probe endobronchial ultrasound.

9. A system, comprising:
an elongate shaft having a proximal end, a distal end, and a lumen extending therethrough;
a penetrating element positioned adjacent to the distal end of the elongate shaft, the penetrating element configured to penetrate tissue;
an ultrasound transducer positioned proximal to a distal tip and adjacent to the distal end of the elongate shaft; and
a power and control unit in electrical communication with the ultrasound transducer.

10. The system of claim 9, wherein the elongate shaft transitions from a first outer diameter to a second outer diameter.

11. The system of claim 10, wherein an inside diameter of the elongate shaft remains constant when the elongate shaft transitions from the first outer diameter to the second outer diameter.

12. The system of claim 11, wherein the inside diameter is between 0.7 and 1.1 millimeters and the outside diameter is between 1.6 and 2 millimeters.

13. The system of claim 9, wherein the elongate shaft transitions from a first wall thickness to a second wall thickness.

14. The system of claim 9, wherein the power and control unit is in electrical communication with the ultrasound transducer via a conductor extending along an outside surface of the elongate shaft.

15. The system of claim 9, wherein the elongate shaft includes two or more lumens.

16. The system of claim 9, wherein the elongate shaft includes one or more visualization markers.

17. The system of claim 9, wherein the ultrasound transducer comprises a convex probe endobronchial ultrasound.

18. A method, comprising:
advancing an ultrasound device through a body lumen to a target location, the ultrasound device comprising:
an elongate shaft having a proximal end, a distal end, and a lumen extending therethrough,
a penetrating element positioned adjacent to the distal end of the elongate shaft, the penetrating element configured to penetrate tissue, and
an ultrasound transducer positioned proximal to a distal tip and adjacent to the distal end of the elongate shaft;
delivering energy to the ultrasound transducer; and
processing an acoustic energy feedback to generate an image.

19. The method of claim 18, wherein the image comprises a 360 degree radial image.

20. The method of claim 19, comprising penetrating tissue at the target location with the penetrating element to obtain a biopsy sample.
